(19) United States
(12) Patent Application Publication
(10) Pub. No.: US 2025/0075167 A1
(43) Pub. Date: Mar. 6, 2025

(54) PROCESSES AND SYSTEMS FOR PREPARING AND DELIVERING A CONDITIONED AQUEOUS MEDIUM TO ONE OR MORE ALGAL GROWTH PONDS

(71) Applicant: NESTE OYJ, Espoo (FI)

(72) Inventors: Cecil CHURN III, Porvoo (FI); Jeffrey KANEL, Porvoo (FI); David BRYANT, Porvoo (FI)

(21) Appl. No.: 18/723,794

(22) PCT Filed: Dec. 30, 2022
(86) PCT No.: PCT/FI2022/050879

§ 371 Date: Jun. 25, 2024

Related U.S. Application Data
(60) Provisional application No. 63/295,551, filed on Dec. 31, 2021.

(30) Foreign Application Priority Data
Mar. 30, 2022 (FI) 20225276

(51) Int. Cl. C12N 1/12; C12M 1/00; C12M 1/34

(52) U.S. Cl. CPC C12N 1/12; C12M 23/18; C12M 41/26; C12M 41/32

(57) ABSTRACT

The present invention relates to the growth of algae, in particular to processes and systems for preparing and delivering an algal growth medium to one or more algal aquaculture ponds that significantly reduces capital and operating costs relative to known processes and systems, and that provides for a uniform algal growth medium that can be delivered to a plurality of algal aquaculture ponds when a plurality of algal aquaculture ponds is present.

### FIELD

The present invention relates to the growth of algae, in particular to processes and systems for preparing and delivering an algal growth medium to one or more algal aquaculture ponds that significantly reduces capital and operating costs relative to known processes and systems, and that provides for a uniform algal growth medium that can be delivered to a plurality of algal aquaculture ponds when a plurality of algal aquaculture ponds is present.

### BACKGROUND AND OBJECTS

There is increasing interest in using algal biomass as a key intermediate for a plethora of sustainable products, such as a source of renewable energy, as a mode to safely and efficiently capture carbon dioxide from the atmosphere for carbon sequestration, and as a renewable source of chemical intermediates. The algae can be grown and harvested to form an algal concentrate. Often, it is preferable to pass the algal concentrate that is produced by the harvester through an extraction process to separate the algal oil from the algal biomass. The algal oil can be a source of valuable products including carotenoids, fatty acids, and other lipids. The remaining algal biomass can also be a source of valuable products, including animal feeds, soil builder and fertilizer, feed for fermentation, and fuel. The cost of the algal aquaculture step is significant, and there is a need to reduce the capital and operating costs associated with this step in order to make the overall process of converting algal biomass into valuable components economically attractive.

One of the key hurdles with algal aquaculture is the necessity to minimize the introduction of algal predators and competitors into algal aquaculture ponds, thereby allowing them to operate more efficiently. Another key hurdle with algal aquaculture is the ability to efficiently and uniformly fertilize one, two or more (e.g. a plurality of) algal aquaculture ponds. A third key hurdle is the ability to efficiently recycle algal growth medium back to the algal aquaculture ponds in order to fully utilize the established algal growth medium. A fourth key hurdle is the ability to steadily and consistently flow algal growth medium into algal aquaculture ponds. A fifth key hurdle is the difficulty of inoculating the algal aquaculture ponds with desired algae. A sixth key hurdle is the ability to control salinity in the algal growth medium by distributing makeup water to offset evaporative losses to one, two or more (e.g. a plurality of) algal aquaculture ponds.

U.S. Pat. No. 4,958,460 offers a system for growth and harvesting of Dunaliella. However, U.S. Pat. No. 4,958,460 fails to describe how the growth medium, recycle streams, nutrients, and the algal inoculum might be delivered to a multiplicity of ponds in an efficient manner. Instead, U.S. Pat. No. 4,958,460 focuses on one system comprised of an optional treatment reservoir followed by a growth reservoir, with interconnecting channels. Transport from the treatment reservoir to the growth reservoir is by a channel; however, U.S. Pat. No. 4,958,460 does not describe how the addition of various streams are blended together, how flow is controlled into the growth reservoir, nor how predators and competitors are controlled.

In a similar approach, U.S. Pat. No. 7,662,616 mentions the importance of recycle streams, but does not disclose how the recycle streams are handled nor how to distribute them to a multiplicity of pond systems. U.S. Pat. No. 7,662,616 also does not provide a solution as to how to minimize competitors and predators inherent in a raw culture medium.

In view of the above, there is a need in the art for improved processes and a system that can overcome the five key hurdles described above so that large-scale algal aquaculture systems can be efficiently and economically operated and controlled.

### SUMMARY

The present invention overcomes the deficiencies of the prior art by providing processes, systems, and uses (of the system) which efficiently condition an aqueous medium, e.g., by adding algal nutrients, salts, and any other suitable materials, to the aqueous medium to provide a conditioned algal growth medium. The conditioned algal growth medium can then be efficiently delivered to one or more algal aquaculture ponds, and in certain embodiments to a plurality of algal aquaculture ponds.

The processes, systems, and uses (of the system) are further capable of minimizing the introduction of algal predators and competitors into one or more algal aquaculture ponds; efficiently and uniformly fertilizing a plurality of algal aquaculture ponds; efficiently recycling algal growth medium back to one or more algal aquaculture ponds in order to fully utilize a conditioned algal growth medium; steadily and consistently flowing algal aquaculture medium into one or more algal aquaculture ponds; efficiently controlling the salinity of the algal aquaculture ponds; and efficiently inoculating one or more algal aquaculture ponds with desired algae.

Advantages of the present invention include, but are not limited to, providing simple processes, aquaculture pretreatment units and systems for solving the problems of ineffective, laborious and not uniform fertilization of one, two or more (e.g. a plurality of) algal aquaculture ponds. Decreased energy consumption and sustainability can also be obtained with the processes and systems of the present invention. Furthermore, one aim of the present invention is to enable steady and consistent flow of well-mixed, homogeneous algal growth medium into one or more algal aquaculture ponds. The present invention enables improving processes and systems for culturing algae, e.g., at a large scale.

Accordingly, in one aspect of the present invention, there is disclosed a process for preparing and delivering a conditioned aqueous medium to one or more algal aquaculture ponds, wherein the process comprises: conditioning an aqueous medium with at least algal nutrients in an aquaculture pretreatment unit to obtain the conditioned aqueous medium, the conditioned aqueous medium capable of promoting growth of algae therein; controlling a gravity flow of the conditioned aqueous medium from the aquaculture pretreatment unit to one or more algal aquaculture ponds; and modifying a salinity of the aqueous medium or the conditioned aqueous medium prior to delivery of the conditioned aqueous medium to the one or more algal aquaculture ponds. The aqueous medium to be conditioned optionally comprises algal cells.

In one embodiment, the controlling of the gravity flow is done via one or more means for controlling flow, or flow controllers, that control flow of the conditioned aqueous medium to the one or more algal aquaculture ponds.

In one embodiment, the one or more flow controllers comprise one or more weirs, and optionally the one or more weirs comprise one or more flow controllers selected from the group consisting of an overflow weir, an underflow or submerged weir (such as an underflow submerged weir), a flow control valve, and combinations thereof.

In one embodiment, the process further comprises filtering the aqueous medium or the conditioned aqueous medium prior to gravity flow of the conditioned aqueous medium to the one or more algal aquaculture ponds or the plurality of algal aquaculture ponds.

In one embodiment, the aquaculture pretreatment unit is elevated relative to the one or more algal aquaculture ponds or has a greater depth than the one or more algal aquaculture ponds to allow for the gravity flow from the aquaculture pretreatment unit to the one or more algal aquaculture ponds. Instead or in addition to a greater depth, the pretreatment unit may have a greater surface elevation than the one or more algal aquaculture bonds or the liquid surface of the one or more algal aquaculture bonds, to allow for the gravity flow. This embodiment is, as the other embodiments, relevant to both the present process and system.

According to an embodiment, a depth to width ratio of the aquaculture pretreatment unit is from 1:100 to 1:2. The depth to width ratio of the aquaculture pretreatment unit can be for example from 1:100, 1:90, 1:80, 1:70, 1:60, 1:50, 1:40, 1:30, 1:20, or 1:10, up to 1:90, 1:80, 1:70, 1:60, 1:50, 1:40, 1:30, 1:20, 1:10, 1:5, or 1:2.

In certain embodiments, an aqueous stream having a high salinity can be fed to the pretreatment unit, in order to control its salinity. High salinity can mean for example saturated salinity.

In an embodiment, the process further comprises modifying a pH of the conditioned aqueous medium prior to delivery of the conditioned aqueous medium to the one or more algal aquaculture ponds.

In an embodiment, the process further comprises recycling a used conditioned aqueous medium from the one or more algal aquaculture ponds or from a harvester located downstream of and in fluid communication with the one or more algal aquaculture ponds for the conditioning step. In a particular embodiment, the used algal aquaculture medium is combined with fresh aqueous medium and the resulting aqueous medium is subjected to the conditioning step.

In an embodiment, the process further comprises continuously flowing the conditioned aqueous medium to the one or more algal aquaculture ponds as an amount of used conditioned aqueous medium is discharged from the one or more algal aquaculture ponds.

In an embodiment, the process further comprises causing turbulent mixing of the aqueous medium with the algal nutrients in the aquaculture pretreatment unit to provide the conditioned aqueous medium. The turbulent mixing is generally a result of a turbulent flow. This is because a turbulent flow occurs when a fluid flows with irregular fluctuations in contrast to laminar flow, in which the fluid moves in smooth paths or layers. For example, with a turbulent flow, the speed of the fluid at a point is continuously undergoing changes in both magnitude and direction, which can cause mixing of components.

In a particular embodiment, the turbulent mixing of the aqueous medium with the algal nutrients is caused by adding the algal nutrients via one, two or more, such as a plurality of, independent nutrient input streams to the aqueous medium as the aqueous medium flows through the aquaculture pretreatment unit. In an embodiment, the plurality of independent nutrient input streams comprises independent input streams for at least two of nitrogen, iron, and phosphorus.

In an embodiment, the process further comprises adding a member selected from the group consisting of seawater, recycled aqueous medium, used conditioned aqueous medium, and inoculum to the aqueous medium in the aquaculture pretreatment unit to generate the conditioned aqueous medium. In a particular embodiment, the adding of a member selected from the group consisting of seawater, recycled aqueous medium, used conditioned aqueous medium, and inoculum causes turbulent mixing of the added member with the aqueous medium.

In an embodiment, the aqueous medium has a residence time in the aquaculture pretreatment unit of from about 0.5 to about 72 hours, preferably about 0.5 to about 48 hours, and more preferably about 0.5 to about 24 hours.

In an embodiment, the aqueous medium comprises a salinity and the aqueous medium is obtained from one or more of a recycled stream, a natural water source, and a man-made reservoir.

In an embodiment, seawater is accumulated in the aquaculture pretreatment unit during elevated tidal periods, or semi continuously, or continuously e.g. from a well such as a Ranney collector well.

In an embodiment, the process may further require reducing an amount of algal competitors and/or algal predators in the aqueous medium or the conditioned aqueous medium by filtration, addition of one or more oxidizing agents, and/or use of gamma irradiation.

In accordance with another aspect, there is provided an algal aquaculture system comprising: one or more algal aquaculture ponds; an aquaculture pretreatment unit configured for conditioning an aqueous medium with at least algal nutrients, wherein the aquaculture pretreatment unit is arranged to feed the conditioned aqueous medium to the one or more algal aquaculture ponds by gravity feed; and a source of a salinity in fluid communication with the aquaculture pretreatment unit to increase a salinity of the aqueous medium or conditioned aqueous medium.

In an embodiment, the system comprises one or more flow controllers arranged to control the feed of the conditioned aqueous medium from the aquaculture pretreatment unit to the one or more algal aquaculture ponds, optionally wherein the one or more flow controllers comprise one or more weirs, and optionally wherein the one or more weirs comprise a member selected from the group consisting of an overflow weir, an underflow weir, a flow control valve, and combinations thereof.

In an embodiment, the system is configured to continuously flow the conditioned aqueous medium to the one or more algal aquaculture ponds as an amount of aqueous medium is discharged from the one or more algal aquaculture ponds.

In one embodiment, the aquaculture pretreatment unit is in fluid communication with a source of the algal nutrients, and the source of the algal nutrients is arranged to cause turbulent mixing of the aqueous medium with the algal nutrients to form the conditioned aqueous medium upon addition to the aquaculture pretreatment unit.

In one embodiment, the source of the algal nutrients comprises one, two, or more, e.g. a plurality of, independent nutrient input streams for adding the algal nutrients to the aquaculture pretreatment unit, wherein optionally the plurality of independent nutrient input streams comprises independent input streams for at least two of nitrogen, iron, and phosphorus.

In one embodiment, the system further comprises a source of a pH modifier in fluid communication with the aquaculture pretreatment unit to increase pH of the aqueous medium or conditioned aqueous medium. In a particular embodiment, the source of additional salinity and/or pH is configured to create turbulent mixing between the aqueous medium or conditioned aqueous medium and the additional salinity and/or pH modifier upon addition to the aquaculture pretreatment unit.

In one embodiment, the one or more algal aquaculture ponds comprise a plurality of algal aquaculture ponds, both in the present process and system.

In an embodiment, the aquaculture pretreatment unit comprises means for transferring the conditioned aqueous medium, such as a conduit, through which the conditioned aqueous medium flows to the one or more algal aquaculture ponds, both in the present process and system. The transfer means can be any suitable means for transferring the process stream from one pond to another. It may for example be a conduit, a pipe, a gutter, a canal, a channel or similar, or any combination thereof. The transfer means can have means for controlling the flow of the process stream, such as a valve, for example at its inlet (i.e. an outlet of the growth pond), at its outlet (i.e. an inlet of the algal aquaculture pond), in between these two or it may comprise several of such controlling means. If need be, the transfer means can also be equipped with a pump or some other equipment for transferring the process stream, while the transfer preferably takes place under gravity, without any external devices.

In an embodiment, the aquaculture pretreatment unit, the one or more algal aquaculture ponds, and/or the one or more flow controllers of the system or process comprise a barrier to water permeation, both in the present process and system.

In an embodiment, the one or more algal aquaculture ponds are open ponds.

In an embodiment, the aqueous medium is conditioned with a used algal aquaculture medium obtained from a bionutrient recovery facility, both in the present process and system.

In an embodiment, the salinity of the aqueous medium or conditioned aqueous medium in the aquaculture pretreatment unit is equal to the salinity of the medium in the one or more algal aquaculture ponds.

In an embodiment, the salinity of the conditioned aqueous medium in the aquaculture pretreatment unit and/or in the one or more algal aquaculture ponds is at least about 7 wt-%, at least about 8 wt-%, at least about 9 wt-%, at least about 10 wt-%, at least about 11 wt-%, at least about 12 wt-%, at least about 13 wt-%, at least about 14 wt-%, at least about 15 wt-%, at least about 16 wt-%, at least about 17 wt-%, at least about 18 wt-%, at least about 19 wt-%, at least about 20 wt-%, at least about 21 wt-%, at least about 22 wt-%, at least about 23 wt-%, at least about 24 wt-%, or at least about 25 wt-%.

In an embodiment, the size of the aquaculture pretreatment unit and/or the one or more algal aquaculture ponds is about 0.1-about 1000 hectares, about 0.1-about 200 hectares, about 0.1-about 100 hectares, about 0.1-about 20 hectares, about 1-about 50 hectares, about 1-about 20 hectares, about 1-about 10 hectares, or about 5-about 10 hectares. For example, the size of the aquaculture pretreatment unit can be about 0.1-about 100 hectares, about 0.1-about 20 hectares or about 1-about 10 hectares, and/or the size of the one or more algal aquaculture ponds can be about 0.1-about 1000 hectares, about 0.1-200 about hectares or about 1-about 50 hectares.

In an embodiment, the algae comprises one or more microalgal species selected from the group consisting of
Amphora sp., Anabaena sp., Anabaena flos-aquae, Ankistrodesmus falcatus, Arthrospira sp., Arthrospira (Spirulina) obliquus, Arthrospira (Spirulina) platensis, Botryococcus braunii, Ceramium sp., Chaetoceros gracilis, Chlamydomonas sp., Chlamydomonas mexicana, Chlamydomonas reinhardtii, Chlorella sp., Chlorella fusca, Chlorella protothecoides, Chlorella pyrenoidosa, Chlorella stigmataphora, Chlorella vulgaris, Chlorella zofingiensis, Chlorococcum citriforme, Chlorococcum littorale, Closterium sp., Coccolithus huxleyi, Cosmarium sp., Crypthecoddinium cohnii, Cryptomonas sp., Cyclotella cryptica, Cyclotella nana, Dunaliella sp., Dunaliella bardawil, Dunaliella salina, Dunaliella tertiolecta, Dunaliella viridis, Euglena gracilis, Fragilaria, Fragilaria sublinearis, Gracilaria, Haematococcus pluvialis, Hantzschia, Isochrysis galbana, Microcystis sp., Monochrysis lutheri, Muriellopsis sp., Nannochloris sp., Nannochloropsis sp., Nannochloropsis salina, Navicula sp., Navicula saprophila, Neochloris oleoabundans, Neospongiococcum gelatinosum, Nitzschia laevis, Nitzschia alba, Nitzschia communis, Nitzschia paleacea, Nitzschia closterium, Nitzschia palea, Nostoc commune, Nostoc flagellaforme, Pavlova gyrens, Peridinium, Phaeodactylum tricornutum, Pleurochrysis carterae, Porphyra sp., Porphyridium aerugineum, Porphyridium cruenturn, Prymnesium, Prymnesium paruum, Pseudochoricystis ellipsoidea, Rhodomonas sp., Scenedesmus sp., Scenedesmus braziliensis, Scenedesmus obliquus, Scenedesmus quadricauda, Scenedesmus acutus, Scenedesmus dimorphus, Schizochytrium sp., Scytonema, Skeletonema costatum, Spirogyra, Schiochytrium limacinum, Stichococcus bacillaris, Synechoccus, Tetraselmis sp., Tolypothrix sp., genetically-engineered varieties thereof, and any combinations thereof; or
one or more prokaryotes selected from the group consisting of Aphanothece halophytica, Microcoleus chthonoplastes, M. lyngbyaceus, Spirulina major, S. platensis, Nodularia spumigena, Dactylococcopsis salina, Synechocystis DUN52, PCC 6803, Synechococcus PCC 7418, Phormidium spp., Oscillatoria spp., Lyngbya spp., Halospirulina tapeticola, Microcystis spp., Nostoc spp., and Aphanocapsa spp.; or
one or more eukaryotes selected from the group consisting of Dunaliella spp., Dangeardinella saltitrix, Chlorella vulgaris, Navicula spp., and Amphora spp.; or
genetically-engineered varieties of any of the above; or
any combinations thereof.

In an embodiment, the algae or microalgae have not been genetically modified or do not originate from genetically engineered algae or microalgae. In a specific embodiment, the algae or microalgae is selected from the group comprising or consisting of Dunaliella sp., Dunaliella bardawil, Dunaliella salina, Dunaliella tertiolecta, Dunaliella parva and Dunaliella viridis, and any combination thereof. In a specific embodiment, the algae or microalgae is Dunaliella salina.

In an embodiment, the one or more algal aquaculture ponds comprises a plurality of algal aquaculture ponds, and the aquaculture pretreatment unit comprises one or more segmented regions to direct or prevent flow of the conditioned aqueous medium into selected ones of the plurality of algal aquaculture ponds.

In an embodiment, any embodiment of a system as disclosed herein is configured to carry out an embodiment of a process as disclosed herein.

In one embodiment of the invention, an influent pump station is used to charge or is for charging the aqueous medium to the aquaculture pretreatment unit and/or an influent pump station is used to charge or is for charging used conditioned aqueous medium, optionally from a bionutrient recovery facility, to the aquaculture pretreatment unit.

According to an embodiment, a flow rate of the aqueous medium (such as a flow rate of sea water) to the aquaculture pretreatment unit is set by the evaporation rate(s) in the one or more algal aquaculture ponds. In such a case, there would be no or at least significantly less need to add further sources of salinity to the aqueous medium.

In one embodiment of the invention, filtration takes place at one or more of the following positions: before one or more influent pump stations, after one or more influent pump stations, before the aquaculture pretreatment unit, before or after the conditioning, in the aquaculture pretreatment unit, after the aquaculture pretreatment unit, in one or more flow controllers, and immediately before the one or more algal aquaculture ponds; or the system comprises one or more filters, optionally at one or more of the following positions: before one or more influent pump stations, after one or more influent pump stations, before the aquaculture pretreatment unit, in the aquaculture pretreatment unit, after the aquaculture pretreatment unit, in one or more flow controllers, and immediately before the one or more algal aquaculture ponds.

In an embodiment, there is disclosed any use of a system as disclosed herein for conditioning an aqueous medium to be used in the growth of algae, treating an algal aquaculture, and/or culturing algae.

In certain aspects, the processes and systems disclosed herein enable an efficient mode of preparing an algal growth medium and introducing the algal growth medium into one or more algal growth ponds while simultaneously and efficiently adding algal nutrients to form an algal growth medium. In certain embodiments, the algal aquaculture pretreatment unit is aligned to feed the multiplicity of algal growth ponds. In an embodiment, the liquid level in the algal aquaculture unit is elevated sufficiently to supply the conditioned aqueous medium by gravity feed to the one or more algal growth ponds. In an embodiment, the algal aquaculture pretreatment unit also controls the flow of the algal growth medium into individual algal growth ponds. In an embodiment, the predators and competitors are controlled via the utilization of filtration equipment at one or more locations.

In an embodiment, the aquaculture pretreatment unit allows acclimation of algae in recycled flows to a change in salinity, and initial growth of seed algae in such recycled flows. In an embodiment, the algal aquaculture pretreatment unit also blends and returns any recycled streams to the one or more algal growth ponds. In an embodiment, selected flows are introduced into the aqueous medium and create turbulence to mix desired materials in the aqueous medium and provide the conditioned aqueous medium, while minimizing the expenditure of energy for mixing.

In one embodiment, there is disclosed a process for an algal aquaculture pretreatment unit wherein: the aqueous medium is conditioned with algal nutrients; it is aligned to feed a multiplicity of algal growth ponds; its liquid level is elevated sufficiently to supply the conditioned aqueous medium by gravity feed to the algal growth ponds; and it controls the flow of the algal growth medium into individual algal growth ponds.

In one embodiment, there is disclosed a process for an algal aquaculture pretreatment unit wherein: the aqueous medium is conditioned with algal nutrients; it is aligned to feed a multiplicity of algal growth ponds; its liquid level is elevated sufficiently to supply the conditioned aqueous medium by gravity feed to the algal growth ponds; and it controls the flow of the algal growth medium into individual algal growth ponds wherein algal competitors and predators are controlled by filtration. Alternatively, filtration could be replaced or combined with the addition of any strong oxidizing agent, such as chlorine, ozone, and/or hydrogen peroxide, or the use of gamma irradiation.

In one embodiment, there is disclosed a process for an algal aquaculture pretreatment unit wherein: the aqueous medium is conditioned with algal nutrients; it is aligned to feed a multiplicity of algal growth ponds; it is elevated sufficiently to supply the conditioned aqueous medium by gravity feed to the algal growth ponds; and it controls the flow of the algal growth medium into individual algal growth ponds wherein reconditioned algal aquaculture media from a bionutrient recovery facility is recycled to the algal aquaculture pretreatment unit.

In one embodiment, there is disclosed a process for an algal aquaculture pretreatment unit wherein: the aqueous medium is conditioned with algal nutrients; it is aligned to feed a multiplicity of algal growth ponds; it is elevated sufficiently to supply the conditioned aqueous medium by gravity feed to the algal growth ponds; and it controls the flow of the algal growth medium into individual algal growth ponds wherein a barrier to water permeation is deployed.

In one embodiment, there is disclosed a process for an algal aquaculture pretreatment unit wherein: the aqueous medium is conditioned with algal nutrients; it is aligned to feed a multiplicity of algal growth ponds; it is elevated sufficiently to supply the conditioned aqueous medium by gravity feed to the algal growth ponds; and it controls the flow of the algal growth medium into individual algal growth ponds wherein the algal aquaculture pretreatment unit comprises one or more segmented regions to facilitate smooth flow of the algal growth medium into the algal growth ponds.

In one embodiment, there is disclosed a process for an algal aquaculture pretreatment unit wherein: the aqueous medium is conditioned with algal nutrients; it is aligned to feed a multiplicity of algal growth ponds; it is elevated sufficiently to supply the conditioned aqueous medium by gravity feed to the algal growth ponds; and it controls the flow of the algal growth medium into individual algal growth ponds wherein ocean water is accumulated during elevated tidal periods.

In one embodiment, there is disclosed a process for an algal aquaculture pretreatment unit wherein: the aqueous medium is conditioned with algal nutrients; it is aligned to feed a multiplicity of algal growth ponds; it is elevated sufficiently to supply the conditioned aqueous medium by gravity feed to the algal growth ponds; and it controls the flow of the algal growth medium into individual algal growth ponds wherein an optional influent pump station is used to charge aqueous medium and optionally reconditioned algal aquaculture media from a bionutrient recovery facility to the algal aquaculture pretreatment unit.

In one embodiment, there is disclosed a process for an algal aquaculture pretreatment unit wherein: the aqueous medium is conditioned with algal nutrients; it is aligned to feed a multiplicity of algal growth ponds; it is elevated sufficiently to supply the conditioned aqueous medium by gravity feed to the algal growth ponds; and it controls the flow of the algal growth medium into individual algal growth ponds wherein filtration is used to control the presence of algal predators and algal competitors wherein the filtration step is located at one or more of the following positions: before the optional influent pump station, e.g. Ranney Collector Well, after the optional influent pump station, and immediately before the algal growth ponds.

In one embodiment, there is disclosed a process for an algal aquaculture pretreatment unit wherein the aqueous medium is charged to the algal aquaculture pretreatment unit by an influent pump station; the aqueous medium is conditioned with algal nutrients; it is aligned to feed a multiplicity of algal growth ponds; it is elevated sufficiently to supply the conditioned aqueous medium by gravity feed to the algal growth ponds; and it controls the flow of the algal growth medium into individual algal aquaculture ponds via weirs.

In one embodiment, there is disclosed a process for an algal aquaculture pretreatment unit wherein the aqueous medium is charged to the algal aquaculture pretreatment unit by an influent pump station; the aqueous medium is conditioned with algal nutrients; it is aligned to feed a multiplicity of algal growth ponds; it is elevated sufficiently to supply the conditioned aqueous medium by gravity feed to the algal growth ponds; and it controls the flow of the algal growth medium into individual algal aquaculture ponds via weirs wherein algal competitors and predators are controlled by filtration.

In one embodiment, there is disclosed a process of an algal aquaculture pretreatment unit wherein the aqueous medium is charged to the algal aquaculture pretreatment unit by an influent pump station; the aqueous medium is conditioned with algal nutrients; it is aligned to feed a multiplicity of algal growth ponds; it is elevated sufficiently to supply the conditioned aqueous medium by gravity feed to the algal growth ponds; and it controls the flow of the algal growth medium into individual algal aquaculture ponds via weirs wherein reconditioned algal aquaculture media from a bionutrient recovery facility is recycled to the algal aquaculture pretreatment unit.

In one embodiment, there is disclosed a process of an algal aquaculture pretreatment unit wherein the aqueous medium is charged to the algal aquaculture pretreatment unit by an influent pump station; the aqueous medium is conditioned with algal nutrients; it is aligned to feed a multiplicity of algal growth ponds; it is elevated sufficiently to supply the conditioned aqueous medium by gravity feed to the algal growth ponds; and it controls the flow of the algal growth medium into individual algal aquaculture ponds via weirs wherein a barrier to water permeation is deployed.

In one embodiment, there is disclosed a process for an algal aquaculture pretreatment unit wherein the aqueous medium is charged to the algal aquaculture pretreatment unit by an influent pump station; the aqueous medium is conditioned with algal nutrients; it is aligned to feed a multiplicity of algal growth ponds; it is elevated sufficiently to supply the conditioned aqueous medium by gravity feed to the algal growth ponds; and it controls the flow of the algal growth medium into individual algal aquaculture ponds via weirs wherein ocean water is accumulated during elevated tidal periods.

In one embodiment, there is disclosed a process wherein: an aqueous medium is charged to an algal aquaculture pretreatment unit by an influent pump station; the aqueous medium is conditioned with algal nutrients; reconditioned growth media from a bionutrient recovery facility is charged to the algal aquaculture pretreatment unit by an influent pump station; it is aligned to feed a multiplicity of algal growth ponds; it is elevated sufficiently to supply the conditioned aqueous medium by gravity feed to the algal growth ponds; and it controls the flow of the algal growth medium into individual algal growth ponds.

### DESCRIPTION OF THE DRAWINGS

FIG. 1 illustrates an algal aquaculture system in accordance with an aspect of the present invention.

FIG. 2 illustrates an algal aquaculture system in accordance with another aspect of the present invention.

FIG. 3 illustrates an embodiment of an aquaculture pretreatment unit in accordance with an aspect of the present invention.

FIG. 4 illustrates an embodiment of an algal aquaculture system of the present invention comprising an aquaculture pretreatment unit and one or more algal aquaculture ponds. Dashed lines show optional components and steps.

FIG. 5 illustrates elevation variations in a mixed tide environment.

FIG. 6 illustrates elevation variations with a semi-diurnal tide.

FIG. 7 illustrates elevation variations in Mexico.

### DETAILED DESCRIPTION

As used herein, “wt-%” refers to a dry mass of a component in a solution in grams divided by 100 grams of the solution. In addition, unless otherwise stated herein or clear from the context, any percentages referred to herein are understood to refer to wt-%.

As used herein, the term “about” refers to a value that is +1‰ f the stated value. In addition, it is understood that reference to a range of a first value to a second value includes the range of the exact stated values, e.g., a range of about 1 to about 5 also includes the more precise range of 1 to 5. Further, it is understood that the ranges disclosed herein include any selected subrange within the stated range, e.g., a subrange of about 50 to about 60 is contemplated in a disclosed range of about 1 to about 100.

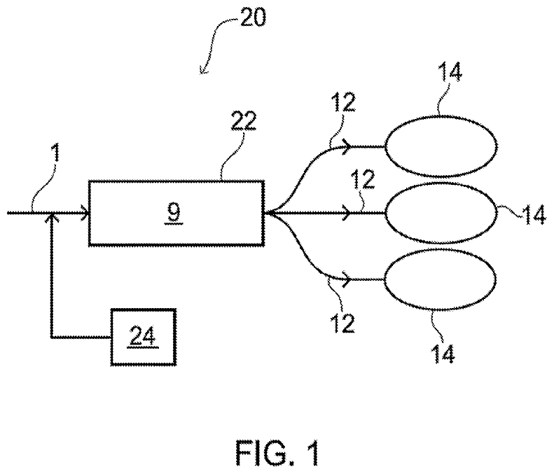

Now referring to the Figures, FIG. 1 illustrates an algal aquaculture system 20 (hereinafter “system 20”) in accordance with the present invention comprising one or more algal aquaculture ponds 14 (hereinafter “pond(s) 14”) and an algal aquaculture pretreatment unit 9. The algal aquaculture pretreatment unit 9 (hereinafter also “pretreatment unit 9” or “aquaculture pretreatment unit 9”) is configured to condition an aqueous medium 1 fed to the pretreatment unit 9 with algal nutrients and any other desired components to generate a conditioned aqueous medium 12. The pretreatment unit 9 is arranged to feed the conditioned aqueous medium 12 to the pond(s) 14 by gravity feed. FIG. 1 illustrates a plurality of the pond(s) 14, but it is understood that the present invention is not so limited. To reiterate, one aspect of the present invention is that the aquaculture pretreatment unit 9 may deliver a uniform conditioned aqueous medium to a plurality of ponds to ensure optimal and uniform algae growth in a plurality of ponds. Typically, one pretreatment unit 9 can be used for several ponds, while each bond preferably has its own exit at the pretreatment unit. In FIG. 1, only one exit is shown for sake of clarity of the Figure.

The pond(s) 14 may be of any suitable shape and size to carry out the desired algae growth in the presence of the conditioned aqueous medium 12. In an embodiment, the size of the pretreatment unit 9 or the pond(s) 14 is about 0.1-about 1000 hectares, about 0.1-about 200 hectares, about 0.1-about 100 hectares, about 0.1-about 20 hectares, about 1-about 50 hectares, about 1-about 20 hectares, about 1-about 10 hectares, or about 5-about 10 hectares. Due to the optional continuous flow of the conditioned aqueous medium 12 to the pond(s) 14 and out of the pond(s) 14 as will be discussed below, large scale ponds may be provided for the mass production of algae and algal biomass. In certain embodiments, the pond(s) 14 comprise or are open ponds.

The pretreatment unit 9 may comprise any suitable structure that allows one or more components to be mixed with the aqueous medium and be output as a mixed (conditioned) aqueous medium. Thus, by “conditioned” it is meant that one or more components may be added and mixed with an aqueous medium. The one or more components may comprise algal nutrients, salts to provide a conditioned aqueous medium 12 with a desired salinity, pH adjusters to provide the conditioned aqueous medium 12 with a desired pH, inoculum to provide the conditioned aqueous medium 12 with seed algae for the growth of algae in the pond(s), or any other desired components, or any combination of the above. Further, in certain embodiments, the aqueous medium 1 has a residence time in the pretreatment unit 9 of from about 0.5 to about 72 hours, preferably about 0.5 to about 48 hours, and more preferably about 0.5 to about 24 hours.

In an embodiment, the pretreatment unit 9 comprises means for transferring, such as a conduit (as explained above), through which the conditioned aqueous medium 12 flows to the pond(s). In one embodiment, the conduit 22 is closed in that it is not open to the environment. In other embodiments, the conduit 22 is open in that the contents are in contact with the environment. In certain embodiments, the conduit 22 may comprise a canal that is provided in the earth leading to the one or more ponds. The conduit 22 may be of any suitable depth, width, and length. In an embodiment, the conduit 22 has a depth of from about 0.2 to about 20 meters, a width of about 0.5 to about 200 meters, and a length of about 5 to about 20,000 meters. It is appreciated that the size of the conduit 22 may be selected based upon a number of factors, including the size of individual ponds, the concentration of materials in the system, the flow rate to each pond, the tide pattern, and the design of any other fluid transport structures in the system.

In accordance with one aspect of the present invention, the components to be mixed with the aqueous medium 1 fed to the aquaculture pretreatment unit 9, e.g., conduit 22, may be added such that they cause turbulence in the aqueous medium 1 sufficient to cause mixing of the added components upon addition to the pretreatment unit 9. In this way, aspects of the present invention reduce capital and operational costs associated with mechanical mixers, e.g., paddle wheels and the like, normally required in such applications. Suitable flow rates for introduction of components to the aqueous medium may be from 1 milliliter per minute to 10 cubic meter per second.

Referring again to FIG. 1, there is thus shown an embodiment of a source of algal nutrients 24 in fluid communication with the pretreatment unit 9 to provide an amount of algal nutrients in the aqueous medium 1. The algal nutrients may be any nutrients suitable for promoting the growth of algae. Without limitation, the algal nutrients may comprise nitrogen, phosphorus, iron, trace mineral nutrients, and combinations thereof. Suitable nitrogen sources include, but are not limited to ammonia, urea, nitrates, or combinations thereof. Suitable phosphorus sources include, but are not limited to phosphoric acid, diammonium phosphate, phosphates, and other sources of phosphorus, or combinations thereof. Suitable iron sources are EDTA chelated iron, and other soluble and insoluble forms of iron. There are a number of further micronutrients that are needed by algae, such as sulphur, manganese, copper, zinc, molybdenum and boron that can be added. In some embodiments, these micronutrients are contained in seawater and other sources of water, and thus may be provided therefrom. In other embodiments, the desired nutrients are mixed in a suitable medium and provided to the source 24 to be fed to the pretreatment unit 9. In still other embodiments, at least a portion of the desired nutrients may be provided from a process stream, e.g. a waste or recycle stream from the growth of aquatic animals that provides at least a portion of the desired nutrients.

Although not illustrated in FIG. 1, it is understood that a source of any other desired component for the conditioned aqueous medium 12, e.g., a source of salts, a pH adjuster, inoculum, or the like may also be in fluid communication with the pretreatment unit 9 to provide the respective component to the aqueous medium.

When salts are added to the pretreatment unit 9, the salts may be in any suitable form for providing a desired salinity in the conditioned aqueous medium 12. In an embodiment, the salts comprise any one or more of sea salts, underground salts, salts of aquifer water, salts of a terminal lake, sodium chloride, and/or any combination of ions present in sea salt. In an embodiment, the amount of salts provided is that effective to provide the conditioned aqueous medium 12 with a salinity of at least about 7 wt-%, at least about 8 wt-%, at least about 9 wt-%, at least about 10 wt-%, at least about 11 wt-%, at least about 12 wt-%, at least about 13 wt-%, at least about 14 wt-%, at least about 15 wt-%, at least about 16 wt-%, at least about 17 wt-%, at least about 18 wt-%, at least about 19 wt-%, at least about 20 wt-%, at least about 21 wt-%, at least about 22 wt-%, at least about 23 wt-%, at least about 24 wt-%, or at least about 25 wt-%. In certain embodiments, a salinity of the conditioned aqueous medium is at least about 7 wt-%, at least about 10 wt-%, or at least about 12 wt-%. In certain embodiments, the conditioned aqueous medium 12 is saturated with salt. In particular embodiments, the salinity of the conditioned aqueous medium 12 is from about 10 wt-% to saturation, about 12 wt-% to saturation, about 20 wt-% to saturation, about 10 wt-% to about 20 wt-%, about 12 wt-% to about 20 wt-%, about 10 wt-% to about 15 wt-%, about 12 wt-% to about 25 wt-%, about 15 wt-% to about 25 wt-%, or about 20 wt-% to saturation.

In certain embodiments, the salinity of the aqueous medium 1 in the aquaculture pretreatment unit 9 is equal to the salinity of the medium in the pond(s) such that the delivered conditioned aqueous medium 12 comprises the desired salinity in the pond(s) 14. In other embodiments, the conditioned aqueous medium 12 traveling from the pretreatment unit 9 may have a lower salinity than that desired in the pond(s) 14 to lower the salinity of the existing conditioned aqueous medium 12 in the pond(s) 14, if the existing conditioned aqueous medium 12 in the pond(s) has a salinity greater than desired. This may occur, for example, due to evaporation of water from the pond(s) 14. In still other embodiments, the conditioned aqueous medium 12 traveling from the pretreatment unit 9 may have a greater salinity than that desired in the pond(s) 14 to increase the salinity of the existing conditioned aqueous medium 12 in the pond(s) 14, if the existing conditioned aqueous medium 12 in the pond(s) has a salinity lower than desired.

In other embodiments, when a pH adjuster is added to the aqueous medium 1 in the pretreatment unit 9 to generate the conditioned aqueous medium 12, the pH adjuster may be any suitable acidic, basic, or neutral component. In one embodiment, the pH of the conditioned aqueous medium 12 is in the range from about 5 to about 10, preferably in the range of about 6 to about 9, and more preferably from about 8 to about 9. In an embodiment, the pH adjuster may be a mineral acid or base that is readily available. Examples of suitable acids or bases include, but are not limited to sodium hydroxide, potassium hydroxide, ammonia or aqueous ammonia or acidic compounds such as phosphoric acid, sulfuric acid, hydrochloric acid or combinations thereof. Alternatively, pH can be maintained by adding a suitable buffering compound such as but not limited to bicarbonate or carbonate, or combinations thereof to the growth media.

In still other embodiments, when inoculum is added to the aqueous medium 1 in the pretreatment unit 9, the inoculum may be any algae species desired to be grown in the pond(s) 14. In an embodiment, the algae comprises one or more microalgal species selected from the group consisting of
Amphora sp., Anabaena sp., Anabaena flos-aquae, Ankistrodesmus falcatus, Arthrospira sp., Arthrospira (Spirulina) obliquus, Arthrospira (Spirulina) platensis, Botryococcus braunii, Ceramium sp., Chaetoceros gracilis, Chlamydomonas sp., Chlamydomonas mexicana, Chlamydomonas reinhardtii, Chlorella sp., Chlorella fusca, Chlorella protothecoides, Chlorella pyrenoidosa, Chlorella stigmataphora, Chlorella vulgaris, Chlorella zofingiensis, Chlorococcum citriforme, Chlorococcum littorale, Closterium sp., Coccolithus huxleyi, Cosmarium sp., Crypthecoddinium cohnii, Cryptomonas sp., Cyclotella cryptica, Cyclotella nana, Dunaliella sp., Dunaliella bardawil, Dunaliella salina, Dunaliella tertiolecta, Dunaliella viridis, Euglena gracilis, Fragilaria, Fragilaria sublinearis, Gracilaria, Haematococcus pluvialis, Hantzschia, Isochrysis galbana, Microcystis sp., Monochrysis lutheri, Muriellopsis sp., Nannochloris sp., Nannochloropsis sp., Nannochloropsis salina, Navicula sp., Navicula saprophila, Neochloris oleoabundans, Neospongiococcum gelatinosum, Nitzschia laevis, Nitzschia alba, Nitzschia communis, Nitzschia paleacea, Nitzschia closterium, Nitzschia palea, Nostoc commune, Nostoc flagellaforme, Pavlova gyrens, Peridinium, Phaeodactylum tricornutum, Pleurochrysis carterae, Porphyra sp., Porphyridium aerugineum, Porphyridium cruentum, Prymnesium, Prymnesium paruum, Pseudochoricystis ellipsoidea, Rhodomonas sp., Scenedesmus sp., Scenedesmus braziliensis, Scenedesmus obliquus, Scenedesmus quadricauda, Scenedesmus acutus, Scenedesmus dimorphus, Schizochytrium sp., Scytonema, Skeletonema costatum, Spirogyra, Schiochytrium limacinum, Stichococcus bacillaris, Synechoccus, Tetraselmis sp., Tolypothrix sp., genetically-engineered varieties thereof, and any combinations thereof; or
one or more prokaryotes selected from the group consisting of Aphanothece halophytica, Microcoleus chthonoplastes, M. lyngbyaceus, Spirulina major, S. platensis, Nodularia spumigena, Dactylococcopsis salina, Synechocystis DUN52, PCC 6803, Synechococcus PCC 7418, Phormidium spp., Oscillatoria spp., Lyngbya spp., Halospirulina tapeticola, Microcystis spp., Nostoc spp., and Aphanocapsa spp.; or
one or more eukaryotes selected from the group consisting of Dunaliella spp., Dangeardinella saltitrix, Chlorella vulgaris, Navicula spp., and Amphora spp.; or
genetically-engineered varieties of any of the above; or
any combinations thereof.

In certain embodiments, the simultaneous addition of two or more components to the aqueous medium 1 is particularly effective to improve mixing of components in the aqueous medium 1. For example, the simultaneous addition of a flow stream comprising nutrients and a flow stream comprising salts may cause mixing of algal nutrients in the aqueous medium 1. In other embodiments, there may be provided multiple streams of the same type, e.g., multiple salt flows or multiple nutrient flows to create the turbulent mixing. In a particular embodiment, for example, the source of algal nutrients 24 may comprise a plurality of independent nutrient input streams for adding algal nutrients to the pretreatment unit 9. In an embodiment, the plurality of independent nutrient input streams comprises independent input streams for at least two of nitrogen, iron, or phosphorus. In this way, for example, nitrogen and iron may be added to the aqueous medium 1 to be mixed in the aqueous medium 1.

In accordance with an aspect of the present invention, the system 20 advantageously reduces or eliminates the need for pumping equipment to deliver the conditioned aqueous medium 12 from the pretreatment unit 9 to the pond(s) 14, thereby significantly reducing capital and operational costs. This may be accomplished by configuring the system 20 such that the conditioned aqueous medium 12 is gravity fed to the pond(s) 14. To provide the gravity fed medium 12, any suitable structure(s) or configurations may be utilized. In one embodiment, the pretreatment unit 9 or the liquid level in the pretreatment unit 9 is elevated relative to the pond(s) 12 or the liquid level in the pond(s) 12 to a degree sufficient to allow gravity flow of the conditioned aqueous medium 12 from the pretreatment unit 9 to the pond(s) 14. In certain embodiments, the pretreatment unit 9 is elevated by 0 to about 10 meters above the mean growth pond elevation. In other embodiments, the gravity feeding of the conditioned aqueous medium 12 to the pond(s) 14 may be done by providing the pretreatment unit 9 with a sufficiently greater depth than the pond(s) 14. In certain embodiments, a ratio of a mean depth of the pretreatment unit 9 to a mean depth of the pond(s) 14 is at least about 1.5:1, about 2:1, about 3:1 or more.

It is understood that the system 20 may include any suitable structures to enable and control flow of materials through the system 20, such as a piping, pumps, flow controllers, computer-based controllers to regulate the flow and/or instrumentation to monitor and/or regulate the composition of materials in the system 20. In one embodiment, the system 20 comprises one or more flow controllers (flow controller(s)), for example, to regulate flow of the conditioned aqueous medium 12 to the pond(s) 14. In such embodiments, the flow controller(s) may be placed at any suitable location upstream of the pond(s) 14, such as at or adjacent an outlet end of the pretreatment unit 9.

The flow controller(s) may comprise one or more of a weir, a sluice gate, a pump, such as a siphon pump, or a flow control valve. In a particular embodiment, the flow controller(s) comprise a weir, such as underflow weir, submerged weir, or an overflow weir. In certain embodiments, the flow controller comprises an underflow weir. In an aspect of the present invention, the underflow weir allows for fluctuation in the liquid level of the pretreatment unit 9 without significantly altering the flow of the conditioned aqueous medium 12 to the pond(s). A continuous and even flow of the conditioned aqueous medium 12 to the pond(s) 14 is desired.

In certain embodiments, the system 20 is configured for continuous operation—meaning that the conditioned aqueous medium 12 may be continuously flowed to the pond(s) 14 as an amount of the conditioned aqueous medium 12 is discharged from the pond(s) 14.

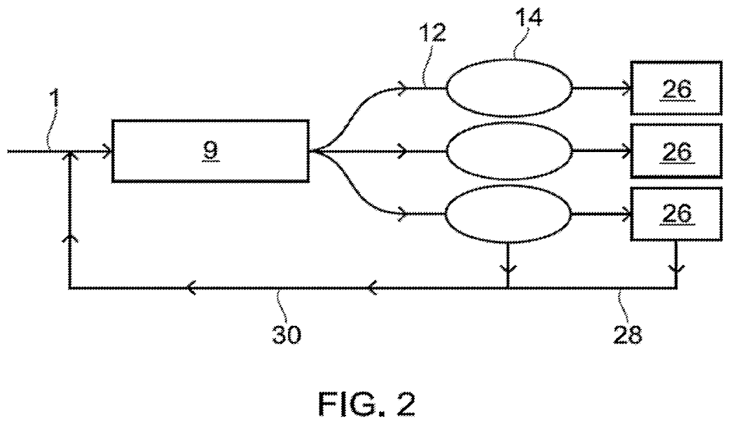

From the pond(s) 14, the conditioned aqueous medium which has been utilized to grow algae may be discharged to any suitable location. In certain embodiments, the conditioned aqueous medium 12 may be directed to an open body of water, such as an ocean, lake, or river, to one or more further growth ponds for further growth of algae, or to one or more harvesters, for the harvesting and collection of algae. Thus, in certain embodiments, the system 20 may further include one or more further ponds (not shown) and/or one or more harvesters 26 (harvester(s) 26) as shown in FIG. 2. Each harvester has means for collecting the harvested algae and preferably also means for recycling any liquid or other products of the pond(s).

In accordance with another aspect, the system 20 is configured for recycling of any used aqueous conditioned medium 28 (hereinafter also “used medium”) from the pond(s) 14 and/or the harvester(s) 26 back to the pretreatment unit 9 for reuse of the used medium 28 in the conditioned aqueous medium 12 delivered to the pond(s) 14. Accordingly, the system 20 may include any suitable structures, such as flow controllers, pumps, fluid lines, and the like to enable the recycling of the aqueous conditioned medium. As shown in FIG. 2, there is provided a recycle line 30 extending from the harvester(s) 26 and/or the pond(s) 14 back to the pretreatment unit 9 for recycling of the used conditioned aqueous medium 28. In one embodiment, the used medium flows by gravity from the harvester 26 to the pretreatment unit 9, via the recycle line 30.

In accordance with another aspect, the aquaculture pretreatment unit 9, the pond(s) 14, flow controllers, harvester(s) 26, or any other component of the system or any combination thereof may comprise a suitable barrier material thereon to prevent water permeation and loss of fluid in the system. Suitable barrier materials include, but are not limited to clays, cement, and suitable materials typically used with liners in aquaculture ponds 14.

In certain embodiments, the aqueous medium 1 in the pretreatment unit 9 is conditioned with a used algal aquaculture medium obtained from a bionutrient recovery facility. The bionutrient recovery facility may recover nutrients from any suitable source, such as shrimp, fish, shellfish, poultry, or other animal waste streams, polishing ponds, aquaculture waste treatment ponds, and the like.

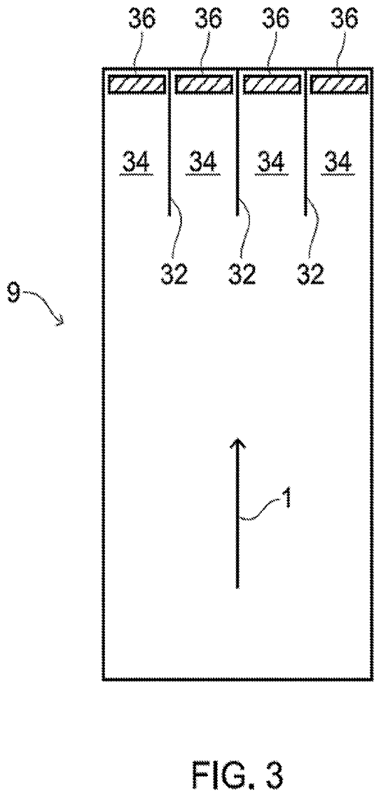

In still other embodiments, there is provided a plurality of the ponds 14, and the pretreatment unit 9 comprises one or more segmented regions configured to direct or prevent flow of the conditioned aqueous medium 12 into selected ones of the plurality of ponds 14. The segmented regions, for example, could include segments 32 that divide the pretreatment unit 9 into a plurality of lanes 34, each of which is in fluid communication with one or more of the ponds 14 (FIGS. 1-3). A weir 36 may be provided at an outlet end of each lane 28 to allow or block flow of the conditioned aqueous medium 12 to the one or more pond(s) 14. Blockage of the flow may be desired, for example, when one or more of the ponds 14 are undergoing maintenance, are being drained, or are otherwise taken out of service.

In accordance with another aspect of the present invention, the systems disclosed herein may be utilized to carry out a process for preparing and delivering the conditioned aqueous medium 12 (algal growth medium) to the pond(s) 14. In an embodiment, the process comprises conditioning the aqueous medium 1, which optionally comprises algal cells, with at least algal nutrients in an aquaculture pretreatment unit 9 to obtain the conditioned aqueous medium 12, wherein the conditioned aqueous medium 12 is capable of promoting growth of algae therein. In an embodiment, the process further comprises controlling a gravity flow of the conditioned aqueous medium 12 from the aquaculture pretreatment unit 9 to the pond(s) 14.

As set forth above, the processes disclosed herein may further include one or more (e.g. all) of the following steps, features, and/or structures for carrying out one or more (e.g. all) of the disclosed steps:
filtering the aqueous medium 1 or the conditioned aqueous medium 12 prior to gravity flow of the conditioned aqueous medium 12 to the pond(s) 14;
modifying a salinity of the conditioned aqueous medium 12 prior to delivery of the conditioned aqueous medium 12 to the pond(s) 14.
modifying a pH of the conditioned aqueous medium 12 within the aquaculture pretreatment unit 9 prior to delivery of the conditioned aqueous medium 12 to the pond(s) 14;
recycling used conditioned aqueous medium 28 from the pond(s) 14 or from one or more harvesters 26 located downstream of and in fluid communication with the pond(s) 14 for the conditioning step;
combining the used conditioned aqueous medium 28 with fresh aqueous medium and subjecting the resulting aqueous medium 1 to the conditioning step;
continuously flowing the conditioned aqueous medium to the pond(s) 14 as an amount of used conditioned aqueous medium 12 is discharged from the pond(s) 14;
causing turbulent mixing of the aqueous medium 1 with the algal nutrients in the aquaculture pretreatment unit 9 to provide the conditioned aqueous medium 12;
causing turbulent mixing of the aqueous medium with the algal nutrients by adding the algal nutrients via one, two or more, such as a plurality of, independent nutrient input streams to the aqueous medium 1 as the aqueous medium 1 flows through the aquaculture pretreatment unit 9;
providing at least two of nitrogen, iron, and phosphorus as the independent nutrient input streams;
adding a member selected from the group consisting of seawater, irrigation water, potable water, recycled aqueous medium, and inoculum to the aqueous medium 1 in the aquaculture pretreatment unit 9 to provide the conditioned aqueous medium 12;
adding of a member selected from the group consisting of seawater, irrigation water, potable water recycled aqueous medium, and inoculum to the pretreatment unit 12 to cause turbulent mixing of the added member with the aqueous medium 9 in the pretreatment unit;
providing the aqueous medium 1 with a residence time in the aquaculture pretreatment unit 9 of from about 0.5 to about 72 hours, preferably about 0.5 to about 48 hours, and more preferably about 0.5 to about 24 hours;
providing an aqueous medium 1 having a salinity from one or more of a recycled stream, a natural water source, and a man-made reservoir.
accumulating seawater in the aquaculture pretreatment unit 9 during elevated tidal periods; and
reducing an amount of algal competitors and/or algal predators in the aqueous medium 1 or the conditioned aqueous medium 12 by filtration, addition of one or more oxidizing agents, and/or exposure to gamma irradiation.

The below description will further elaborate on features of the systems, uses of the systems, and processes disclosed. It is understood that an open pond aquaculture system for the cultivation of marine microalgae relies on a saline or hypersaline medium supplied with sunlight, and proper nutrients. Algal aquaculture ponds (algal growth ponds) can be designed to optimize retention times for the desired algal product. Enhancement of the system or process of the present invention may be achieved by the ability to feed one, two, three, four, five, six, seven, eight, nine, ten or more, e.g. a multiplicity of, algal aquaculture ponds with a preconditioned and well-blended algal growth medium.

In accordance with aspects of the present invention, aqueous medium may be obtained from a saline source and conveyed to an optional influent pump station via a canal, lined or unlined, or by pipeline. When the source of aqueous medium is the ocean, competitors and predators of the chosen microalgae can be removed by filtration associated with a Ranney collector well when a pipeline is utilized for media conveyance, or a sand filter when either an inlet canal or pipeline is utilized. When an inlet canal to an influent pump station conveys the media at high tides, an optional first filtration step may precede the pump station. Alternatively, the optional influent pump station may lift the aqueous medium to an optional second filtration step consisting of or comprising e.g. a sand filtration, sock filter, rotary drum filter, multiple screens, or combinations thereof. Following pumping, nutrients may be supplemented along with any recycle streams, such as the flow from a bionutrient recovery facility, thereby taking advantage of the turbulence from the pump discharge for blending all streams. The use of turbulent mixing minimizes the overall energy input.

The blend of aqueous medium may be directed to the aquaculture pretreatment unit 9 as described herein, and from this point forward may flow by gravity to the algal growth pond(s) 14. The pretreatment unit 9 may further function to further blend recycled flows, inoculum from an algal seed nursery, and supplemented nutrients for distributing it in a controlled manner to a multiplicity of algal growth ponds. The pretreatment unit 9 may be exposed to sunlight to allow seed algae to acclimate to higher salinities, and possibly begin to grow and replicate, thereby minimizing any lag in the growth of algae in the pond(s) 14, which would require more hydraulic retention time to accommodate.

In certain embodiments, the pretreatment unit 9 may be either a man-made or a naturally occurring channel that can be lined or unlined (to minimize costs). The channel can be subdivided into segments meant to aid in the storage of extra media to cover when the influent pumping station is unable to pump the total daily requirement of aqueous medium.

In one embodiment, flow control out of the pretreatment unit 9 into the pond(s) 14 may be done by weirs. Weirs can be of several designs, but preference is for a submerged flow control weir to regulate the flow into each of the algal growth ponds 14 by one or more flow control structures (flow controllers) per pond. The flow into the algal growth ponds can pass through an optional third filtration process comprising a series of screens built into the flow control structures or filter socks of decreasing mesh size attached to the discharge side of the flow control structures.

The pond(s) 14 may be comprised of open ponds or raceways that are not mixed or mixed, or a combination of these. Mixing is typically done by a paddlewheel, but other mixing devices applicable to shallow ponds and raceways may be utilized. Typically, a paddlewheel is preferred as it is gentler and needs only a low horsepower motor to turn it at the required rpm. The pond(s) 14 and/or raceways can be lined or unlined depending on the soil type. Open raceway ponds are usually oval, or serpentine in design with one or more paddlewheels or mixing devices employed to move the culture in a loop throughout the pond(s) 14. Smaller raceways and ponds may be covered, but preference is given to being uncovered for gas exchange with the atmosphere.

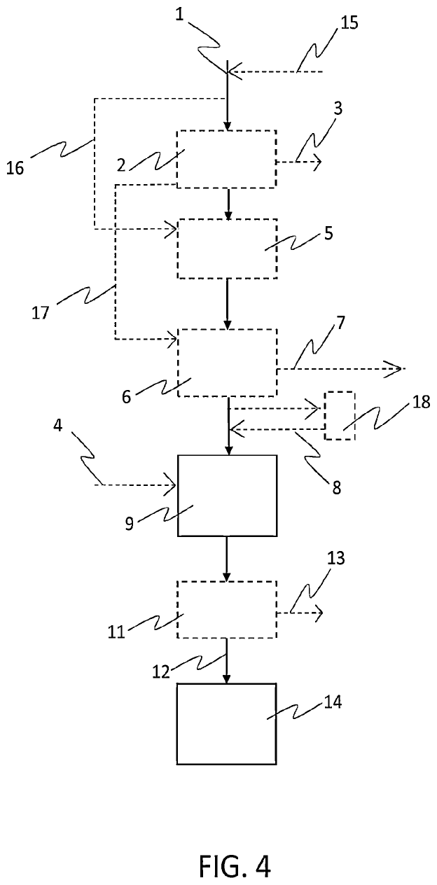

A further embodiment of a system in accordance with the present invention is shown in FIG. 4. In the Figure, optional flows and equipment are indicated by dashed lines.

The aqueous medium (Stream 1 in FIG. 4) entering the algal aquaculture pretreatment unit 9 may be obtained from one or more sources, including, but not limited to an ocean, a sea, a lake, a river or stream, an underground aquifer, a canal, an irrigation canal, a wastewater discharge, effluent from an aquaculture facility such as from a shrimp farm or from a fish farm, or combinations thereof. The aqueous medium 1 may comprise water, mineral salt, heavy metals, microalgae, algal predators, algal competitors, and residual nutrients.

If the aqueous medium 1 comprises mineral salt, in certain embodiments, the salinity of the aqueous medium 1 used to feed the aquaculture pretreatment unit 9 is less than the highest salinity experienced by the algae in the algal growth ponds (or bioreactors) 14. This is due to the fact that water in the aqueous medium 1 is replacing water that evaporates from the aquaculture pretreatment unit 9 and the algal growth ponds 14.

When the culture medium's salinity is equal to or greater than seawater, then the system is preferably located near the ocean, sea, or other source of saline water, so that valuable fresh water is not required for the algal aquaculture system. When located near the ocean or sea, there will typically be an inlet canal 15 allowing ocean or seawater to feed a tidal pool area that will serve as the feed for an influent pump station. The inlet canal 15 can have any suitable form, for example as described above for the transfer means.

The concentration of copper in the aqueous medium 1 is critical to control, and the preferred concentration of copper is less than 2×10⁻⁶ Molar, and more preferred less than 8×10⁻⁷ Molar. Avron and BenAmotz in “Dunaliella: Physiology, Biochemistry, and Biotechnology”, Chapter 4, CRC Press, Boca Raton, FL (1992) teach the toxicity levels of various metals for several Dunaliella species, the contents of which are incorporated herein by reference. It is preferable to stay below these levels as well, in order to facilitate the most efficient algal growth characteristics.

The aqueous medium 1 may be derived from an ocean or from a sea. The mineral salt content of the aqueous medium 1 is preferably below that in the algal growth ponds 14. Many elements, including heavy metals, are required in trace amounts for optimum algal growth. Copper, for example, is necessary for the production of plastocyanin, a protein involved in electron transport in photosynthesis. Most algae exhibit some degree of inhibition to heavy metals depending on the algae type, concentration of the metal, the pH, and the concentration of chelators (naturally occurring or supplemented). The pH may be important as it determines how much of the metal is present as a free ion, which is typically the more toxic form. Chelators of metals are interesting as they can prevent toxicity by making some metals non-bioavailable, while others, for example iron, can be made more bioavailable with chelation.

The aqueous medium 1 may also be derived from lakes, rivers, streams, or combinations thereof. The lakes may comprise fresh water or saline water, such as that typically found in terminal lakes such as the Great Salt Lake in Utah. The rivers and streams may comprise different levels of mineral salt, heavy metals, algal predators, and algal competitors. It is preferable that the free cupric ion level in the aqueous medium 1 derived from lakes, rivers, and/or streams be as low as possible, and preferably less than twice that found in ocean water.

The aqueous medium 1 may further be derived from underground aquifers provided that the chemical composition of the aqueous medium is conducive to algal growth. It is preferable that aqueous medium derived from underground aquifers have a free cupric ion level less than twice that in seawater, since copper is a known algaecide. When the aqueous medium 1 is derived from an underground aquifer, it is also preferable that the divalent ion concentration be sufficiently low so that the algal growth rate is not negatively impacted by the presence of divalent ions including, but not limited to magnesium, calcium, and combinations thereof. Algae also tend to bioaccumulate heavy metals, such as arsenic that can substitute for phosphorus. Thus, it is preferred that the concentration of arsenic and other heavy metals is less than twice that found in seawater. Open ocean water typically has an arsenic concentration of 1-2 micrograms l⁻¹ (μg/l) while freshwater sources can be up to 10 micrograms l⁻¹ which is United State's Environmental Protection Agency's (EPA) water quality standard limit for drinking water.

The aqueous medium 1 derived from one or more of the previously mentioned sources may comprise water, mineral salts, arsenic, algal predators, and algal competitors. The aqueous medium 1 may also comprise residual nutrients.

The water in the aqueous medium 1 is necessary to facilitate a growth medium for the algae to be grown in the algal growth ponds 14. In an embodiment, water is added to 1) supply a medium in which the algae can grow, and 2) offset water losses due to evaporation from the algal growth ponds 14 and from the aquaculture pretreatment unit 9, 3) offset water losses due to permeation losses of water from the algal growth ponds 14 through the soil, and/or 4) to increase the depth in the aquaculture pretreatment unit 9 and/or the algal growth ponds 14.

The amount of water required each day may depend upon several factors: salinity targets within the aquaculture pond system, the wind speed and the relative humidity. Daily evaporation rates may be a major factor in the determination of daily water needs for the aquaculture system. If evaporation rates are high, then the salinity of the ponds will increase and water will be required to make up for the losses and to maintain the salinity target for each pond. Wind tends to accelerate evaporation rates, and thus the higher the wind speed the higher the evaporation rates for a given salinity. The amount of moisture in the atmosphere is termed the relative humidity, and the lower this value the higher the evaporation rates when all other factors are constant. Consequently, the rate and amount of evaporation directly influences the daily demand for water.

The mineral salts in the aqueous medium 1 may comprise any cations and anions that are present in ocean water. These mineral salts include but are not limited to major cations such as sodium, magnesium, potassium, and calcium and major anions such as chloride, sulfate, bromide, carbonate, bicarbonate, and nitrate. In one embodiment of the instant invention, the mineral salts in the aqueous medium 1 will comprise those typically found in ocean water, as disclosed in Table 1. In another embodiment of the instant invention, the mineral salts in the aqueous medium 1 will comprise those typically found in solar salt evaporation ponds for the production of sodium chloride and other salts of interest. In yet another embodiment of the instant invention, the mineral salts in the aqueous medium 1 will comprise those found in terminal lakes, such as the Great Salt Lake in Utah, the Dead Sea in Israel, Lac Rose in Senegal, Laguna Colorada altiplano region near Potosi, Bolivia, and the Pink Lake near Esperance, West Australia. In yet another embodiment of the instant invention, the mineral salts in the aqueous medium 1 will comprise those found in saline aquifers, specifically those found in the southwest United States, as disclosed in U.S. Pat. No. 6,986,323, the contents of which are incorporated herein by reference, in particular paragraphs [0070]-[0076].

TABLE 1
Concentration of various elements in ocean water
(Produced from data supplied in: Summerhayes,
C. P., and Thorpe, S. A., 1996: Oceanography
An Illustrated Guide, Chapter 11, pp 165-181.)

| | Average | | Units |
|---|---|---|---|
| Element | Concentration | Range | (per kg) |
| Lithium | 174 | (a) | μg |
| Boron | 4.5 | (a) | mg |
| Carbon | 27.6 | 24-30 | mg |
| Nitrogen (b) | 420 | >1-630 | μg |
| Fluorine | 1.3 | (a) | mg |
| Sodium | 10.77 | (a) | g |
| Magnesium | 1.29 | (a) | g |
| Aluminum | 540 | <10-1200 | ng |
| Silicon | 2.8 | <0.02-5 | mg |
| Phosphorus | 70 | <0.1-110 | μg |
| Sulfur | 0.904 | (a) | g |
| Chlorine | 19.354 | (a) | g |
| Potassium | 0.399 | (a) | g |
| Calcium | 0.412 | (a) | g |
| Manganese | 14 | 5-200 | ng |
| Iron | 55 | 5-140 | ng |
| Nickel | 0.5 | 0.1-0.7 | μg |
| Copper | 0.25 | 0.03-0.4 | μg |
| Zinc | 0.4 | <0.01-0.6 | μg |
| Arsenic | 1.7 | 1.1-1.9 | μg |
| Bromine | 67 | (a) | mg |
| Rubidium | 120 | (a) | μg |
| Strontium | 7.9 | (a) | mg |
| Cadmium | 80 | 0.1-120 | ng |
| Iodine | 50 | 25-65 | μg |
| Cesium | 0.29 | (a) | μg |
| Barium | 14 | 4-20.0 | μg |
| Mercury | 1 | 0.4-2 | ng |
| Lead | 2 | 1-35 (c) | ng |
| Uranium | 3.3 | (a) | μg |

(a) Variations are determined entirely or largely by those in salinity, i.e., the element is essentially conservative. For these elements, the average concentration is given for seawater having a salinity of 35 parts per thousand.
(b) Concentrations refer to combined nitrogen; element occurs also as dissolved nitrogen (N₂) gas. Species other than NO₃— are often important in the upper ocean (e.g., NO₂⁻, NH₄⁺).
(c) Concentrations are affected by inputs to the ocean surface by atmospherically transported lead from combustion of leaded petroleum.

Notes: Based mainly upon information in Bruland. Usual ranges for oceanic waters are shown; concentrations of certain elements can be higher in some coastal waters. Chemical oceanographers often employ molar units instead of the mass units shown here. For sodium (atomic weight 22.99), the concentration given above of 10.77 g/kg can alternatively be expressed as 0.468 mol/kg.

The aqueous medium 1 typically comprises algal predators and algal competitors, both of which are commonly deleterious to the productivity of an algal aquaculture. Since the aqueous medium 1 may be derived from a variety of sources as described previously, the exact types of algal competitors and algal predators will differ from source to source.

The algal competitors comprise organisms that compete with the desired algae for nutrients, such as nitrogen, phosphorus, iron, trace nutrients, and combinations thereof. Algal competitors may be algae that do not produce the desired product, or they may be bacteria, diatoms, and combinations thereof. Algal competitors will be specific to the type of algae that is to be grown and the conditions under which it is grown. In one embodiment, when Dunaliella salina is the target algae to be grown, competitors include, but are not limited to Dunaliella parva, D. viridis, and D. minuta. In another embodiment, when Haematacoccus is being grown, competitors include, but are not limited to these same Dunaliella species as well as other marine phytoplankton. In yet another embodiment, when Chlorella is being grown, competitors include, but are not limited to other freshwater or marine algae depending on the environment the chosen Chlorella species is grown in. In general, the types of competitors of consequence will depend on the salinity used in the algal growth ponds.

Salinity is a term that defines the total amount of dissolved inorganic solids (salts) in an aqueous solution. The typical salts found in natural waters may include sodium chloride, calcium and magnesium sulfates, bicarbonates, and carbonates. It is a standard practice to express salinity as parts per thousand (‰), which is not a true percent but an approximation of the milligrams of salt per gram of water. In more general terms, salinity is indicated by the water source, such as a freshwater, a brackish water, a saline water, and a brine. Ranges of salinity are associated with these general terms and these ranges are defined as <0.5‰ (<0.05%) for freshwater, 0.5-30‰ (0.05-3%) for brackish water, 30-50‰ (3-5%) for saline (seawater) water, and >50‰ (>5%) for a brine.

At low salinities, freshwater to seawater, the types of competitors include, but are not limited to, freshwater and marine microalgae, diatoms and cyanobacteria. At intermediate salinities of seawater, about 3.5%, to low salinity brines, about 15%, the types of competitors include, but are not limited to those microalgae that can survive this intermediate range of salinity. At elevated salinities of brines, greater than about 15% sodium chloride, the types of competitors include, but are not limited to any hypersaline microalgae.

The algal predators comprise organisms that prey on the algae as a food source. Algal predators will be specific to the type of algae that is to be grown. In one embodiment of the instant invention, when Dunaliella salina is the target algae to be grown, predators include, but are not limited to the flagellate form of Heteramoeba sp., Fabrea salina and Euplotes sp. In another embodiment of the instant invention, when Haematacoccus is being grown, predators include, but are also not limited to the flagellate form of Heteramoeba sp., Fabrea salina and Euplotes sp. as well as Artemia salina. In yet another embodiment of the instant invention, when Chlorella is being grown, predators include, but are not limited to various protozoans, ciliates, and amoebae.

In general, the types of predators of consequence will depend on the salinity used in the algal growth ponds. At low salinities various protozoa such as Fabrea salina and the amoeba Heteroamoeba sp. can attack a culture and rapidly decimate the algal population. In salinities associated with ocean water, the types of predators include, but are not limited to ciliates, brine shrimp (Artemia), zooplankton, and oligochaetes. At intermediate salinities of 3.5 wt-% to 15 wt-%, the types of predators include, but are not limited to those organisms mentioned that can adapt readily to this higher salinity range. At elevated salinities, greater than 15 wt-% sodium chloride, the types of predators include, but are not limited to only those that can tolerate and reproduce in hypersaline environments. Algal predators include but are not limited to brine shrimp or Artemia sp., the ciliate, Fabrea salina (median length is 200 microns), and combinations thereof.

The aqueous medium 1 may comprise residual nutrients that may be derived from a variety of sources, including, but not limited to agricultural drainage, aquaculture waste streams, livestock and poultry waste streams, effluent from wastewater treatment systems, and combinations thereof. The presence of nutrients in the aqueous medium is preferable, as it reduces the amount of nutrients required to meet the target values in the conditioned aqueous medium 12.

One acceptable source of residual nutrients for use in the present invention is that obtained from agricultural drainage (if pesticide/herbicide levels are acceptable), especially when it comprises significant levels of residual algal nutrients, including, but not limited to nitrogen fertilizers, phosphorus fertilizers, iron fertilizers, trace nutrient fertilizers, and combinations thereof. The use of agricultural drainage as a source of aqueous medium is preferred when the supply flow rate and nutrient concentrations are steadily or easily monitored and controlled.

Another acceptable source of residual nutrients is that obtained from aquaculture waste streams. These aquaculture waste streams may be derived from the husbandry of fish, shrimp, shellfish, and other marine organisms, where the effluent stream from the operation comprises biological waste from the organisms as well as unused fertilizers or feeds. In one embodiment, the aquaculture waste stream from shrimp aquaculture is utilized to supplement the flow and composition of the aqueous medium. In another embodiment, the aquaculture waste stream from fish aquaculture, such as catfish, tilapia, or salmon, is utilized to supplement the flow and composition of the aqueous medium.

Another acceptable source of residual nutrients in the instant invention is that obtained from livestock and poultry waste streams. These waste streams may comprise components that supply compounds that serve as nitrogen and phosphorus fertilizers. It is preferable that this source of residual nutrients be stable throughout the year. If the supply is not relatively constant, then it is preferable to implement a nutrient monitoring system that would facilitate adjustment of the nutrient level to meet the nutrient needs in the algal growth ponds.

Yet another acceptable source of residual nutrients is that obtained from the effluent from wastewater treatment systems. These effluent streams may have varying degrees of treatment (combinations of pretreatment, primary treatment, secondary treatment, and tertiary treatment), and thus may comprise a wide range of nitrogen, phosphorus, or other nutrients. In certain embodiments, the effluent is disinfected via a suitable process to avoid introducing pathogens to the aquaculture.

In certain embodiments, the aqueous medium 1 is optionally passed through a first filtration system 2 in order to reduce the number of algal predators and algal competitors. Since the algal predators and algal competitors are typically larger in size than the microalgae being grown in the algal growth ponds 14, filtration methods are preferred to accomplish the separation. Suitable filtration equipment for the optional first filtration step includes, but is not limited to Ranney Collector wells, deep bed filtration, sand filtration, mixed media filtration, pressure filters, cartridge filters, sock filters, rotary drum filters, and combinations thereof. The retentate comprise the algal predators and algal competitors and is shown as reference numeral 3 in FIG. 4.

In one embodiment, a Ranney Collector well is used to produce the conditioned aqueous medium 12 that is depleted in algal predators and algal competitors. The Ranney Collector well may be utilized to reduce the algal predators and algal competitors from various sources of the aqueous medium, including, but not limited to the ocean, sea, lakes, rivers, streams, and combinations thereof. The description of a Ranney Collector well was first described in U.S. Pat. No. 2,740,476 (in particular in the figures and their description), and more recently in U.S. patent application Ser. No. 11/599,495 (publication US 2007/108112, in particular paragraphs [007] and [033]-[047]) and Ser. No. 11/599,498 (publication US 2007/108133, in particular paragraphs [007] and [027]-[036]), the contents of all are incorporated herein by reference.

Alternatively or additionally, it is possible to control predators and competitors by an oxidation step somewhere in the overall process. Using the aquaculture pretreatment unit 9 for the addition and/or delivery of the oxidizing agent would be a first line of defense. Any strong oxidizing agent will be effective and can be used, but preference is given to chlorine. Chlorine can be delivered as an industrial grade solution, which is typically 10 to 12 wt-%, or a gas, or as a hypochlorite salt. Chlorine is also the preferred oxidizing agent for recovering from a crash of the culture. A crash, the sudden death of the majority of the algal culture, can occur and the addition of chlorine to the media aids in recovery of the algal growth ponds.

Any suitable structures or processes known in the art may be used to transport the aqueous medium 1 to the optional influent pump station 5, including but not limited to canals, pipes, pipelines, ditches, and combinations thereof. Since the flow rate of the aqueous medium 1 can be relatively high, canals are well-suited as the transportation mode. An inlet canal 15 is typically encountered when the aqueous medium 1 is derived from the ocean or sea. The inlet canal 15 should be of a depth and width to provide adequate flow during high tides to the intake of the influent pumps that are mounted on the influent pump station 5 used to transport the aqueous medium 1 into the aquaculture pretreatment unit 9. In some cases, the aqueous medium 1 may be supplied at an elevation so that the optional influent pump station 5 is not required. For example, if the aqueous medium 1 is derived from an irrigation canal, its elevation may be sufficient to obviate the need for the optional influent pump station 5. In another case, the aqueous medium 1 is derived from the ocean only at high tides, where the elevation of the tide is sufficient to eliminate the need for a pump station in order to feed the aquaculture pretreatment unit 9. However, in certain embodiments, the optional influent pump station 5 is present.

The aqueous medium 1 can be transported, via conduit 16, from it source to an optional influent pump station 5, which is used to lift the aqueous medium 1. Preferably, the elevation of the aqueous medium 1 in the algal aquaculture pretreatment unit 9 is sufficiently above the tidal fluctuation so as to allow the aqueous medium 1 to flow by gravity to the algal growth ponds 14.

In certain embodiments, the aqueous medium 1 may pass through an influent pump station 5 that is used to increase the elevation of the conditioned algal growth medium 1 being fed to the algal growth ponds 14. However, in some cases, the aqueous medium 1 may be at a sufficient elevation so that it can flow by gravity into the algal aquaculture pretreatment unit 9 without passing through the influent pump station 5.

Optionally, reconditioned algal aquaculture media 4 from a harvester (not shown in FIG. 4) may be charged to the aquaculture pretreatment unit 9, either directly or via the optional influent pump station 5. In an embodiment, the reconditioned algal aquaculture media 4 is at a sufficient elevation that the optional influent pump station 5 is not needed. The reconditioned algal aquaculture media 4 may comprise water, mineral salts, and microalgae. Acceptable sources of the reconditioned algal aquaculture media 4 include but are not limited to the tails from a harvester downstream of the pond(s) 14, the discharge from the bionutrient recovery facility, and combinations thereof. The tails from the harvester is the stream depleted of algae that exits the harvesting unit. The bionutrient recovery facility is designed to allow bacteria to transform organic molecules in the aquaculture media or the wastewater from shrimp and fish aquaculture back into nutrients that the algae can utilize before the discharge from the bionutrient recovery facility is recycled to the algal growth ponds 14 by way of the aquaculture pretreatment unit 9.

The influent pump station 5 may provide the necessary lift and volume of aqueous medium 1 for the algal growth ponds 14 designed to grow the algal biomass. In an embodiment, the influent pump station 5 provides the primary lift of the aqueous medium 1 and optionally the reconditioned algal aquaculture media 4 to allow the majority of flows to be by gravity from that point forward throughout the system. The influent pump station 5 may comprise one or more pumps that are capable of lifting the aqueous medium 1 and optionally the reconditioned algal aquaculture media 4 into the aquaculture pretreatment unit 9. Any type of pump known in the art can be used to accomplish this lift, including, but not limited to Archimedes screw pumps, centrifugal pumps, diaphragm pumps, other pumps designed to handle large volumetric flow rates, and combinations thereof.

The pumps at the influent pump station 5 may be driven by any power supply that is readily available at that location, including, but not limited to electric power, solar power, tidal power, diesel fuel, fuel oil, algal oil, vegetable oils, biodiesel, gasoline, ethanol, butanol, wind, and combinations thereof.

In one embodiment, the inlet canal 15 allows ocean water or seawater to travel from its source to the influent pump station 5. When the aqueous medium 1 is derived from either of these sources, tidal influences add a complicating factor to the design of the influent pump station 5. The inlet canal 15 will preferentially be earthen-lined, but it could be lined with a plastic liner, or concrete. Pipes could also be used to carry the flow from the ocean to the tidal pool area at the pump suction for the influent pump station 5, but this would be the least desirable unless it is associated with a Ranney Collector well.

In certain embodiments, it may be desirable to pump the aqueous medium 1 into the aquaculture pretreatment unit 9 at times when the elevation of the aqueous medium 1 is the greatest in the suction of the influent pump station 5. High tides occur over several hours, and it is during this time that the influent pump station 5 will be operated to lift the aqueous medium 1 and optionally the reconditioned algal aquaculture media 4 into the aquaculture pretreatment unit 9.

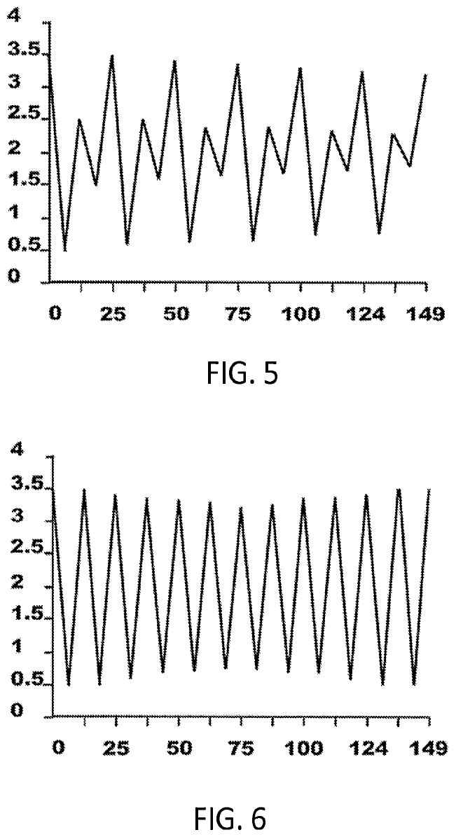

For both mixed tide (FIG. 5, with time in hours on the x-axis and height in meters on the y-axis) and semidiurnal tide (FIG. 6, with time in hours on the x-axis and height in meters on the y-axis) situations there are two high and two low tides per tidal day. However, in a few areas, the rotation of the earth and a continental land mass prevents two high and two low tides per tidal day. In these areas, there is only one high and one low tide per tidal day. In this case, the high tide is usually not as high, but it is spread out over a longer time period and is referred to as a diurnal tide. The northern and western Gulf Coast area of the United States experiences diurnal tides. In these cases, careful attention to the high and low tides levels and the increase in time for operating the influent pump station 5 should be of priority so that the size and number of influent pumps is properly designed for the influent pump station 5.

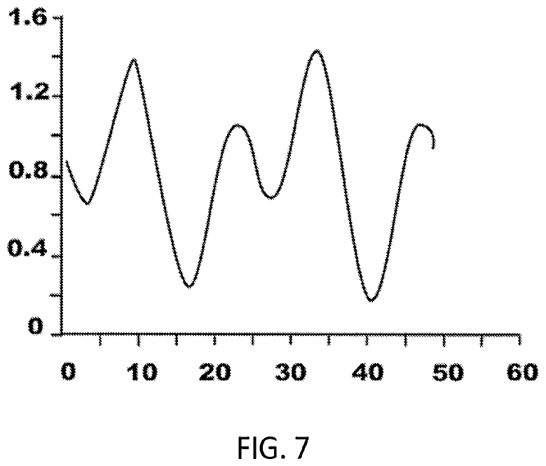

Pumping of seawater can begin as soon as the tidal pool feeding the influent pump station 5 is deep enough to allow pumping. For diurnal tide areas, this will occur once a tidal day, whereas for semi-diurnal and mixed areas it will occur twice a tidal day. In the mixed tide area, the benefit of reduced head at high tides will not be as great during the low high tide, however the tidal pool could be designed to also pump during the high low tide and therefore be able to pump three times during the tidal day. An example of a mixed tide is illustrated in FIG. 7 (with time in hours on the x-axis and tidal elevation in meters on the y-axis) and this particular curve is typical of the west coast of Mexico.

Pumping occurs during elevated tidal periods which fluctuate with the phase of the moon and seasonally. Pumping can begin when the tidal pool feeding the pump station or the algal pretreatment reactor directly, is deep enough to start the pumps. Once the peak of the high tide has passed there will continue to be enough depth of water in the tidal pool to maintain pumping for some time and until the depth becomes too shallow for the pumps to safely pump water. The period of pumping is defined as the elevated tidal period. Pumping can continue to the high-water level of the high tide as well as beyond the slack point (the point that the tide begins to recede to a low water level or low tide). Depending on the time of year and phase of the moon, the strength of the tide will vary, and the pumping times will have to be adjusted, but preferentially pumping will begin 3 to 4 hours prior to the high-water level of the high tide and continue for 3 to 4 hours past this point.

Pumping may be performed with every high tide and therefore every two pumping periods of a semi-diurnal or mixed tide area will need to deliver to the aquaculture pretreatment unit 9, all the water required for evaporative losses from the algal growth ponds 14 over the next 24 hours plus any reserves that are required to maintain normal operations during times of insufficient flows. In the case of an optional first filtration step or unit 2, the high aqueous medium level may be shifted from the actual high tide, due to a lag time in the hydraulic flow as it passes through the filtration unit process.

The discharge from the optional influent pump station 5 may pass through an optional second filtration unit 6 in order to reduce the number of predators and competitors entering the aquaculture pretreatment unit 9. Any filtration equipment known in the art may be used for this separation, including, but not limited to sand filters, sock filters, mixed media filters, rotary drum filters, and combinations thereof. In one embodiment, sock filters may be attached to the discharge from the pumps in the influent pump station 5 in order to capture predators and competitors, as well as other debris. In another embodiment, the discharge from the influent pump station 5 is passed through a sand filter to capture and remove predators and competitors. The retentate comprises the algal competitors and the algal predators, and this stream is identified by reference numeral 7 in FIG. 4.

The aqueous medium 1 can also pass directly from the first filtration unit 2 to the second filtration unit 6, via conduit 17.

The sand bed may be flooded with brine during non-use times to osmotically shock any undesirable organisms trapped in the sand. A simple underdrain system may be employed to make it drain easily into the receiving aquaculture pretreatment unit 9. A sand filter can also contain a layer of iron oxide pellets to remove trace amounts of arsenic from the seawater should this element be a problem in downstream products obtained from the algae. Such dual media sand filters may be used for special purposes depending on the media placed in the unit process.

In certain embodiments, the aquaculture pretreatment unit 9 may comprise a multifunctional canal that is used to transport the conditioned aqueous medium 12 from its source to the multiplicity of algal growth ponds 14. In an embodiment, the aquaculture pretreatment unit 9 is constructed of clayey soils. However, it may be lined with a plastic liner or lined with clay, or a combination thereof in order to mitigate degradation of the structure due to fluid flow or loss of any aqueous medium due to permeation in the soil. In an embodiment, the hydraulic conductivity should be less than 10⁻⁶ to 10⁻⁷ cm sec⁻⁶.

The aquaculture pretreatment unit 9 may serve a dual function as the primary distribution canal to all the algal growth ponds 14 in the system, as well as being a multi-functional reactor. In one embodiment, the aquaculture pretreatment unit 9 may also serve as a reservoir of aqueous medium 1 during times of non-pumping at the influent pump station 5, for example during low tides. Not only will the aqueous medium 1 be pumped into the algal aquaculture pretreatment unit 9 for storage and delivery, but the aquaculture pretreatment unit may also be used to:
i) recycle culture media, with or without seed algae;
ii) add, mix, and deliver fresh nutrients to the algal growth ponds (e.g., nitrogen and phosphorus sources, an iron source, and possibly bicarbonate and/or carbonate);
iii) return reconditioned algal aquaculture media to the algal growth ponds as another potential source of seed algae;
iv) return brine that has gone through a bionutrient recovery step (see FIG. 4);
v) remove predators and competitors by filtration and increasing salinities; and/or,
vi) allow acclimation and initial growth of any seed algae.

In certain embodiments, the delivery of harvester “tails” and treated brine may provide a seed of microalgae to the aquaculture pretreatment unit 9. Given the residence time in the aquaculture pretreatment unit 9 containing adequate nutrients, there will be preliminary acclimation to lower salinities required in the algal growth ponds 14, and growth of this seed.

Algal nutrients may comprise a nitrogen fertilizer system, a phosphorus fertilizer system, an iron fertilizer system, trace nutrient fertilizer system, optionally a carbon source (inorganic or organic carbon), and combinations thereof. The algal nutrients may be added to the pretreatment unit 9 or the optional influent pump station 5, or to the aqueous medium 1 prior to the aquaculture pretreatment unit 9, or to the optional reconditioned algal aquaculture media 4, or combinations thereof. In an embodiment, the algal nutrients are added to the pretreatment unit 9 for distribution to the algal growth ponds 14. In a particular embodiment, the algal nutrients 8 may be added after the optional second filtration step 6 so that no fertilizers are lost in that separation step. The algal nutrients may come from a bionutrient recovery facility 18 or from another source. In one embodiment, the algal nutrients 8 are added to the discharge from the influent pump station 5 to facilitate mixing of the nutrients with the aqueous medium 1, and to reduce overall energy requirements for mixing.

A preferred carbon source is atmospheric carbon dioxide but could be carbon dioxide generated from any combustion source, e.g., coal-fired power plants, or carbon derived from an inorganic carbon source such as a carbonate mineral, including, but not limited to sodium carbonate, sodium bicarbonate, and other bicarbonate and carbonate sources. The carbon source may also be a carbonate solution or gaseous carbon dioxide derived from the atmosphere by any capture method know in the art, such as adsorption or absorption.

The conditioned algal growth medium (conditioned aqueous medium) employed for cultivating microorganisms may also contain other algal growth factors, such as vitamins or growth promoters, which may comprise biotin, riboflavin, thiamine, folic acid, nicotinic acid, pantothenate, pyridoxine, and combinations thereof.

All algal nutrients may be added continuously or batch-wise, depending on the operation of the influent pump station 5, and the ability to use the discharge from the influent pump station to mix the algal nutrients. In an embodiment, for the addition of new fertilizers, they will be stored as aqueous solutions in above ground tanks, with each kept separate from the others. If liquid ammonia is utilized, then it will have a pressure container and an evaporator to add it to water. In one embodiment, the proper dose of each fertilizer will be calculated frequently and will be added to the pretreatment unit 9 using a tank and metering pump or a closed tank and a Mariotte siphon to maintain a constant head and hence a constant flow out of the tank. The nitrogen fertilizer system may comprise organic and/or inorganic nitrogen compounds or materials, which contain these compounds.

Suitable components in the nitrogen fertilizer system include but are not limited to: ammonia in liquid or gaseous form; ammonium salts such as ammonium sulfate, ammonium chloride, ammonium phosphate, ammonium carbonate or ammonium nitrate; nitrates; urea; amino acids; complex nitrogen sources such as corn steep liquor, soybean meal, soybean protein, yeast extract, and meat extract; other nitrogen sources known in the art; or combinations thereof. In an embodiment, the nitrogen fertilizer system will be selected in order to use nitrogen that is readily available locally at a favorable cost. However, it should also be a source of nitrogen that is readily soluble in various salinities, bio-available, and non-toxic. Ammonia is readily soluble but can increase pH if the culture is not buffered. Buffering can be important to prevent acidification when the ammonia is taken up by the algae. Also, as pH and temperature increase, more of the ammonia is present as free (unionized) ammonia and this form can be toxic to the microalgae when in high enough concentrations. Likewise, the amount of free ammonia from the use of ammonium salts such as ammonium acetate, ammonium chloride and ammonium nitrate is a function of pH and temperature. Urea is a potential candidate as it undergoes hydrolysis in water to form ammonium carbonate that acts as a buffer. However, when urea is metabolized the release of ammonia will again be partitioned into the ionized and free forms depending on the pH and temperature.

The phosphorus fertilizer system may comprise any type of phosphorus fertilizer known in the art, with phosphate being the best source of phosphorus. Phosphorus fertilizers include, but are not limited to phosphoric acid, potassium dihydrogen phosphate, dipotassium hydrogen phosphate, super triple phosphate, monoammonium phosphate, diammonium phosphate, phosphoric acid, or the corresponding sodium-containing salts, or combinations thereof. Components in the phosphorus fertilizer system must be chosen carefully to prevent precipitation of phosphorus so that this fertilizer is not lost as solids or made biologically unavailable throughout the aquaculture pretreatment unit 9 and/or in the pond(s) 14. For this reason, the presence of calcium in super triple phosphate means this fertilizer is not a preferred source of phosphorus.

The iron fertilizer system may comprise chelated iron, iron metal that is rusting, iron filings or particles that dissolve in the aqueous medium, or combinations thereof. Chelating agents can be added to the medium in order to keep the metal ions in solution. Particularly suitable chelating agents include dihydroxyphenols such as catechol or protocatechuate, or the disodium salt of ethylenediaminetetraacetate (EDTA), or for alkaline conditions the more stable trisodium hydroxyethylenediaminetriacetate, and even the less effective organic acids such as citric acid and nitrilotriacetic acid. EDTA complexes with iron and prevents its precipitation with phosphates. This also allows a more gradual release of the iron and maintains its bioavailability. Iron deficiencies can reduce the electron carrying capability of the electron transport chain of the photosynthetic apparatus, and to compensate for a reduction in ferridoxin the algae typically produces more flavodoxin, a non-iron containing electron transporter, and an indicator of an iron deficiency. The optimum concentration for iron in a growth medium for D. salina and D. viridis lies between 1.25 to 3.75 mg 1-1 (Borowitzka, 1990).

Maintaining target nutrient levels in the conditioned algal growth medium is a key activity for ensuring successful operation of the aquaculture pretreatment unit 9. When nutrients are present in either the aqueous medium 1 or the optional reconditioned algal aquaculture media 4, a method of measuring the concentration of the algal nutrients may be deployed so that the desired quantity of algal nutrients for algal growth in the algal growth ponds 14 can be added.

This can be achieved via use of an on-site laboratory. The on-site laboratory may analyze frequent samples taken throughout the entire aquaculture system for nitrogen, phosphorus, and iron. Ammonia nitrogen can be detected by an ion-selective electrode or colorimetric methods, nitrate by an enzymatic protocol that produces a color change in relation to the amount and measured spectrophotometrically, and the iron can be measured by an inductively coupled plasma (ICP) method that may be performed at an off-site laboratory. By monitoring residuals, correct supplementation of nutrients will be possible in the algal pretreatment unit 9.

The pH of the aqueous medium in the algal aquaculture pretreatment unit 9 may be conditioned to facilitate optimal growth in the algal growth ponds 14. In one embodiment, the pH is in the range from about 5 to about 10, preferably in the range of about 6 to about 9, and more preferably from about 8 to 9. In an embodiment, the pH is set to the desired level by the addition of a mineral acid or base that is readily available. Examples of suitable acids or bases include, but are not limited to sodium hydroxide, potassium hydroxide, ammonia or aqueous ammonia or acidic compounds such as phosphoric acid, sulfuric acid, or combinations thereof. Alternatively, pH can be maintained by adding a buffering compound to the growth media. In this embodiment, the buffer would supplement the alkalinity of the media through the carbonic acid/bicarbonate/carbonate system. The addition of sodium bicarbonate is preferred as D. salina can utilize bicarbonate due to the presence of an extracellular enzyme, carbonic anhydrase.

Introducing oxygen or oxygen-containing gas mixtures such as, for example, ambient air into the culture will maintain aerobic conditions. Any method known in the art may be deployed in the aquaculture pretreatment unit 9 or in the algal growth ponds 14 to introduce oxygen or oxygen-containing gas mixtures, including, but not limited to paddle wheel aerators, pump-sprayer aerators, propeller aspirator pump aerators, airlift aerators, Venturi mixers or jet eductors, or any of a variety of subsurface diffusers, and combinations thereof.

From freshwater to brackish to marine to hypersaline ecosystems, photosynthesis produces energy-rich bonds and respiring organisms, e.g. bacteria, catalyze redox processes. When these ecosystems are in a steady state photosynthetic production, P is in balance with heterotrophic respiration, R.

P=R, or P/R=1 Equation 1:

An aquaculture for growing a desired microalgae can be operated in balance of P and R; however, knowledge of the complexity of such an ecosystem can be utilized to control it to result in a desired product.

Photosynthesis can be represented by the following stoichiometry:

nCO₂+nH₂O⇔(CH₂O)n+nO₂ Equation 2:

106CO₂+16NO₃⁻+HPO₄²⁻⁻+122H₂O+18H⁺⇔{C₁₀₆H₂₆₃O₁₁₀N₁₆P₁}+138O₂ Equation 3:

106CO₂+16NH₄⁺+HPO₄²⁻⁻+108H₂O⇔{C₁₀₆H₂₆₃O₁₁₀N₁₆P₁}+107O₂+14H⁺ Equation 4:

The above equations illustrate that the nitrogen fertilizer system may comprise either ammonium or nitrate ions; however, one releases hydrogen ions while the other consumes hydrogen ions. Either source of nitrogen may be added to the main canal to allow dispersion into the water before entering the growth ponds. The sodium salt of nitrate or the chloride of ammonia or ammonia may be used for nitrogen supplementation. Of these three options, it is preferred that ammonia be the primary source of any nitrogen supplementation due to its low cost. It is further recognized that the use of ammonia will require vigilance in regard to pH and temperature at the various ammonia doses to prevent a toxic level of free ammonia from being generated. The source of this ammonia can be liquid anhydrous ammonia or preferentially the ammonia found in the daily discharge of a nearby shrimp or fish aquaculture. These discharges can be seasonal and may have to be supplemented with liquid ammonia or other nitrogen source. Some pretreatment to remove any heavy particulate material may be necessary at various times when solids or turbidity are an issue during the peak-growing season for the shrimp or fish. The phosphorus fertilizer system provides phosphorus, which is a necessary macronutrient, like nitrogen, and is preferably supplemented as an orthophosphate salt in the same manner as the nitrogen. Preferably the phosphorus fertilizer system comprises a potassium salt such as potassium mono- or dihydrogen phosphate, or phosphoric acid. However, the phosphorus found in the shrimp or fish aquaculture discharge is also preferred as the source of the phosphorus fertilizer system. The same conditions and limitations apply to phosphorus as they do for nitrogen when using the “natural” source from the shrimp or fish.

The iron fertilizer system provides another important nutrient that is required at micro (<1 mg l⁻¹) amounts. A small amount of iron is necessary for optimal growth and can be added as a salt. Iron (II) or iron (III) salts can be used, but iron (III) is preferred. In addition, iron easily precipitates with phosphate so an addition should be in a chelated form, typically with ethylenediaminetetraacetate (EDTA) and added to the algal aquaculture pretreatment unit remotely from where the phosphate is added.

Equations 3 and 4 above illustrate an excellent starting point on the amount of algal nutrients that are required, and it is referred to as the Redfield ratio. The ratio of carbon in the algal cell to the amount of nitrogen is 106:16, and likewise the ratio of carbon to phosphorus is 106:1. By estimating the amount of algal biomass to be produced, based on time of year (season), solar irradiance, water temperature, the initial seed of biomass, and the anticipated doubling rate of the algae, the amount of carbon can be calculated in the biomass to be grown daily. Using the ratio of C:N:P of 106:16:1 or the unique ratio that is relevant to the specific algae being grown, the amount of nutrients required to grow this biomass can be calculated and added daily to the pretreatment unit 9. In some instances, a slight excess of nutrients may be desired to facilitate monitoring.

The reconditioned algal aquaculture media from a bionutrient recovery facility 18 may comprise the desired microalgae that are to be grown in the algal growth ponds 14, and its recycle to the algal aquaculture pretreatment unit may be used to overwhelm the growth of any unwanted algal species that may be present in the aqueous medium. In this manner, the aquaculture pretreatment unit 9 functions as an inoculum for the desired algae in the algal growth ponds. The addition of this algal inoculum can be anywhere along the length of the aquaculture pretreatment unit 9, but preference is to add it close to the influent pump station for maximum residence time. The bionutrient recovery facility 18 can be used to monitor the nitrogen, phosphorus, and iron levels in the stream feeding the aquaculture pretreatment unit 9, thereby controlling the flow of nutrients.

Optionally, an inoculum of the desired algae may be maintained in one or more algal growth ponds 14 that may feed the aquaculture pretreatment unit 9 to facilitate rapid recovery from any potential crashes in the algal growth ponds 14. This inoculum may be grown in enclosed raceways, greenhouses, raceways, open ponds, tubes, or other bioreactors that would be sufficiently protected from potential environmental threats as to provide a ready source of the desired algae.

The aquaculture pretreatment unit 9 may comprise one or more segments to facilitate smooth flow of the algal growth medium into the algal growth ponds 14. The various segments of the aquaculture pretreatment unit 9 may communicate by any suitable means, such as by additional pumps or by weirs, or combinations thereof. Any type of pumps and hydraulic control structures known in the art may be appropriate to supply this communication. Suitable hydraulic control structures include, but are not limited to rectangular weirs, V-notched weirs, Cipoleti weirs, proportional weirs, submerged weirs, and combinations thereof. For a rectangular weir, the inlet weir will be set at the height required to deliver the required flow to the pond. Adding stop logs to the hydraulic control structure will set the height of the inlet weir, which will control the amount of flow into an individual segment in the aquaculture pretreatment unit 9.

The aquaculture pretreatment unit 9 may feed the conditioned aqueous medium 12 from the aquaculture pretreatment unit 9 to the algal growth ponds 14 by using any method known in the art. In an embodiment, hydraulic control structures will be used to regulate the flow of the conditioned algal growth medium into individual algal growth ponds 14. Suitable hydraulic control structures include, but are not limited to rectangular weirs, V-notched weirs, Cipoleti weirs, proportional weirs, submerged weirs, and combinations thereof. For a rectangular weir, the inlet weir will be set at the height required to deliver the required flow to the pond. Adding stop logs to the hydraulic control structure will set the height of the inlet weir, which will control the amount of flow into an individual pond.

Optional use of a submerged orifice will conserve head and regulate flow into the algal growth ponds. The flow would typically, but not necessarily, be perpendicular to the orifice meaning the orifice will most likely be lateral to the algal pretreatment unit flow.

The size of the algal growth ponds 14 may necessitate more than one hydraulic flow control structure for proper distribution of the conditioned aqueous medium 12 across a single algal growth pond 14. In this case, the number of hydraulic flow control structures communicating between the aquaculture pretreatment unit 9 and a specific algal growth pond 14 should be selected to meet desired hydrodynamic specifications known in the art.

After the pretreatment unit 9, an optional third filtration unit 11 may be used to further control the introduction of algal predators and algal competitors into the algal growth ponds 14. The algal competitors and algal predators are captured in the retentate, and this stream is shown in FIG. 4 as Stream 13. In one embodiment, screening in the pretreatment unit 9 at the inlet of each algal growth pond 14 may be employed. For example, a three-screen series of decreasing mesh sizes located at the weir entrance to the algal growth ponds 14 can be used to exclude algal competitors and algal predators. However, in this embodiment, a significant amount of time and labor may be required to keep the small screens clean to prevent a decrease in the hydraulic flux through the flow control structure. To increase the surface area of the screens, a filter sock can be fabricated of a length and diameter to provide significant surface area and all the flow for each algal growth pond passes through a dedicated sock. An individual sock can be up to several hundred feet long and even up to several hundred yards long depending on the anticipated flow. When the sock becomes clogged with captured algal predators and algal competitors, it can be pulled out of the inlet area, turned inside out, sun-dried, cleaned and reused. The final mesh size in the series of screens or in the filter sock should preferably be <100 micron, but each situation will be different, and the optimum mesh size may be larger or smaller.

The pretreatment unit 9 and pond(s) 14 described herein may be utilized to grow a multitude of microalgae in a culture media of a full range of salinities, ranging from freshwater microalgae to halotolerant microalgae, such as Dunaliella salina.

Algae from the Divisions Chlorophycophyta, Phaeophycophyta, Chrysophycophyta, Cyanophycophyta, Cryptophycophyta, Pyrrhophycophyta and Rhodophycophyta, which are adaptable to saline water as a growth medium, are all suitable for use in the present invention.

More specifically, the algal biomass may comprise any microalgal species (including diatoms, coccolithophorids and dinoflagellates) one desires to separate from the growth medium. These microalgal species include, but are not limited to Amphora sp., Anabaena sp., Anabaena flos-aquae, Ankistrodesmus falcatus, Arthrospira sp., Arthrospira (Spirulina) obliquus, Arthrospira (Spirulina) platensis, Botryococcus braunii, Ceramium sp., Chaetoceros gracilis, Chlamydomonas sp., Chlamydomonas mexicana, Chlamydomonas reinhardtii, Chlorella sp., Chlorella fusca, Chlorella protothecoides, Chlorella pyrenoidosa, Chlorella stigmataphora, Chlorella vulgaris, Chlorella zofingiensis, Chlorococcum citriforme, Chlorococcum littorale, Closterium sp., Coccolithus huxleyi, Cosmarium sp., Crypthecoddinium cohnii, Cryptomonas sp., Cyclotella cryptica, Cyclotella nana, Dunaliella sp., Dunaliella bardawil, Dunaliella salina, Dunaliella tertiolecta, Dunaliella viridis, Euglena gracilis, Fragilaria, Fragilaria sublinearis, Gracilaria, Haematococcus pluvialis, Hantzschia, Isochrysis galbana, Microcystis sp., Monochrysis lutheri, Muriellopsis sp., Nannochloris sp., Nannochloropsis sp., Nannochloropsis salina, Navicula sp., Navicula saprophila, Neochloris oleoabundans, Neospongiococcum gelatinosum, Nitzschia laevis, Nitzschia alba, Nitzschia communis, Nitzschia paleacea, Nitzschia closterium, Nitzschia palea, Nostoc commune, Nostoc flagellaforme, Pavlova gyrens, Peridinium, Phaeodactylum tricornutum, Pleurochrysis carterae, Porphyra sp., Porphyridium aerugineum, Porphyridium cruenturn, Prymnesium, Prymnesium paruum, Pseudochoricystis ellipsoidea, Rhodomonas sp., Scenedesmus sp., Scenedesmus braziliensis, Scenedesmus obliquus, Scenedesmus quadricauda, Scenedesmus acutus, Scenedesmus dimorphus, Schizochytrium sp., Scytonema, Skeletonema costatum, Spirogyra, Schiochytrium limacinum, Stichococcus bacillaris, Synechoccus, Tetraselmis sp., Tolypothrix sp., and genetically-engineered varieties or combinations (mixtures, or mixed cultures) of these microalgal species.

Even more specifically, the algal biomass may comprise any microalgal species (including diatoms, coccolithophorids and dinoflagellates) and including but not limited to the following: Amphora sp., Ankistrodesmus, Arthrospira (Spirulina) plantesis, Botryococcus braunii, Chlamydomonas sp., Chlamydomonas reinhardtii, Chlorella protothecoides, Chlorella sp., Closterium sp., Cosmarium sp., Crypthecoddinium cohnii, Cyclotella sp., Dunaliella salina, Dunaliella tertiolecta, Haematococcus pluvialis, Hantzschia sp., Nannochloris sp., Nannochloropsis sp., Navicula sp., Neochloris oleoabundans, Nitzschia sp., Phaeodactylum tricornutum, Scenedesmus sp., Schiochytrium limacinum, Stichococcus sp., Tetraselmis suecica, and Thalassiosira pseudonana, and genetically-engineered varieties or combinations (mixtures, or mixed cultures) of these microalgal species.

A more preferred group of algae are those with flagella, cilia and/or eyespots. Flagella are a tail-like projections that protrude from the cell body of certain algae and functions in locomotion. Cilia are an adaptation that allows independent cellular creatures, like algae, to move around in search of food. Photosensitive eyespots are found in some free-swimming unicellular algae. Photosensitive eyespots are sensitive to light. They enable the algae to move in relation to a light source. Such algae have the capability of independent motion, phototaxis, and can move towards the surface during daylight. Phototaxis is the movement of microalgae in response to light. For example, certain algae (e.g. D. salina) can perceive light by means of a sensitive eyespot and move to regions of higher light concentration to enhance photosynthesis.

The aquaculture pretreatment unit 9 may supply algal growth ponds 14 that comprise an algal culture that is either a monoculture (primarily only one microalga) or a mixed culture of two or more microalgae. As with any open pond system, there may be competing algae. In one embodiment, the algal growth ponds 14 are operated at an elevated salinity so that few competitive algae can compete during normal operation. The ability to increase salinity to control a competitor is an advantage of the hypersaline system; however, other control strategies can be utilized to control competing algae in low salinity or freshwater systems.

More than one type of marine species may be cultured using the same water from the ocean or aquifer of the invention. This type of serial culturing method provides an opportunity for the culturing of marine species, optionally wherein the waste of one species serves as a nutrient source for the other marine species. For example, shrimp or fish being cultured are fed a predetermined feed, and the waste generated by the fish becomes a nutrient source for the algae. The use of artificial fertilizers for algae can be minimized or avoided by culturing algae in series with fish or shrimp aquaculture.

Marine species must be cultured under conditions (i.e., salinity and temperature) that are compatible with those species. In one embodiment, an aquaculture system for culturing fish or shrimp prior to the culturing of algae comprises a recirculation system where water from a pond containing fish or shrimp waste is optionally passed through a mechanical filter that removes particulate waste. The filtered water is then used as the culture media for microalgae. The microalgae remove phosphorus in the form of dissolved phosphates, and nitrogen in the form of ammonia, nitrites, and nitrates.

The design of this aquaculture system renders it useful for more than just seawater and brine salinities, which allows it to be used for freshwater and brackish water salinities. A series of ponds comprising the pretreatment unit 9, optional filtration, and the algal growth pond may be used as a stand-alone biological waste treatment system or in serial conjunction with more aggressive biological waste treatment systems, e.g. activated sludge, to polish the effluent of residual nitrogen and phosphorus. The source of the nutrients can be either human wastes or animal wastes. The species of algae in these cases would be native species that are grown in either a mono-culture, or as a mixed culture of two or more algal species.

In an aspect of the present invention, algae may be grown in any type of algal growth pond known in the art, including but not limited to fermentation units, enclosed photobioreactors, open-pond bioreactors, and combinations thereof. Many types of algal bioreactors have been proposed in the art, and the subject is currently an area of intense research. Suitable algal bioreactors generally fall into three categories: fermentation units, enclosed photobioreactors, and open-pond bioreactors. The fermentation units are commonly considered for the growth of genetically modified algae that are heterotrophic. The fermentation unit is typically constructed of steel and involves sophisticated process control. This type of algal bioreactor is appropriate for high-value products, such as docosahexaenoic acid (DHA). However, this type of bioreactor is extremely expensive for the production of lower-value chemicals, such as biofuels because of the enormous capital cost of the fermentation equipment. Furthermore, the fermentation process will typically require a source of sugar, which adds costs, especially when lower-value products are being produced.

Enclosed photobioreactors that are transparent so that the algae they contain can utilize the sunlight have also been proposed for the production of biofuels. These enclosed photobioreactors may comprise plastic bags, glass and plastic tubes, ponds in green-house structures, and the like. Tubular reactors were popularized by GreenFuel Technologies Corporation of Cambridge, Massachusetts for the production of biofuels, but the technology was economically unsuccessful. Plastic bag bioreactors are typified by those utilized by Solix Biofuels of Fort Collins, Colorado. Although the capital cost of constructing a bioreactor from plastic instead of steel is substantially reduced, this type of bioreactor is still so expensive that the only commercial use is for the production of a high value product such as astaxanthin, a carotenoid. Thus, the use of enclosed photobioreactors is only of commercial interest for the production of high-value products.

Open-pond bioreactors are generally classified as natural, intensive, and extensive, and this type of bioreactor is preferred for use with the instant invention. The natural open-pond bioreactors are defined as those naturally occurring ponds where the conditions are right to grow algae. These ponds may contain either fresh or saline water, and they are unmanaged in terms that they lack controlled fertilizer addition and mechanical agitation. Natural open ponds that contain algae are common along the shores of the Great Salt Lake in Utah.

Both the intensive and extensive modes of aquaculture require the controlled addition of fertilizers to the medium in order to supply the necessary nutrients, such as phosphorus, nitrogen, iron, and trace metals, that are necessary for biomass production through photosynthesis. The primary difference between the two modes of production is mixing of the growth medium. Intensive ponds employ mechanical mixing devices while extensive ponds rely on happenstance mixing. Therefore, factors that affect algae growth can be more accurately controlled in intensive aquaculture.

Intensive aquaculture ponds are frequently constructed of concrete block and are lined with plastic. Brine depth generally is controlled at about 20 centimeters, which has been considered to be the optimum depth for producing algal biomass. A number of configurations of these ponds have been proposed. However, the open-air raceway ponds are typically the most important commercially. Raceway ponds employ paddle wheels to provide mixing especially for algal cultures growing a non-motile alga. Chemical and biological parameters are carefully controlled, including salt and fertilizer concentrations, pH of the growth media, and purity of the culture.

Extensive aquaculture has been practiced in the hot and arid regions of Australia for the production of beta-carotene. Outdoor ponds for extensive aquaculture generally are larger than those for intensive aquaculture and normally are constructed in lakebeds, coastal tidal flats and coastal plains. The open-air ponds are typically bounded by earthen dikes. No mechanical mixing devices are employed.

Algae growth ponds that utilize these types of aquaculture systems, others known in the art, and combinations thereof, may be used with the instant invention.

### Example 1

Seawater, nutrients, and various recycle streams including any algae inoculum were all blended in an algal aquaculture pretreatment unit for distribution into one or more algal growth ponds. Dunaliella salina algae grow in algal growth ponds that are open ponds, and an individual pond can cover two to 100 hectares. The algal growth medium was removed from the algal growth ponds by pumps and fed to an adsorptive bubble separation unit where the algal biomass was dewatered to form an algal concentrate. The algal growth medium depleted of algae, referred to as the tails were either returned to the algal aquaculture pretreatment unit directly for reuse or sent to a bionutrient recovery facility before the reconditioned algal aquaculture media was returned to the algal aquaculture pretreatment unit. A slipstream of the tails was sent to an evaporation pond as a purge stream for the excess mineral salt in the overall aquaculture system.

### Example 2

Fresh water, phosphoric acid, urea, ammonia, sodium carbonate, and chelated iron, were added directly to the algal aquaculture pretreatment unit. The fresh water was added through a 3 inch diameter pipe that discharged 60 centimeters above the liquid level in the algal aquaculture pretreatment unit so that all of the components were well mixed. The tails stream from an adsorptive bubble separation unit (harvester) that was depleted in water was also added at the same location as the fresh water so that all of the components were well mixed as they entered the algal aquaculture pretreatment unit. This unit fed two algal growth pond systems that were each two hectares of wet surface area. The amount of fresh water added was calculated based on the evaporation rate from the four hectares of ponds. The amount of nitrogen and phosphorus fertilizers were added based on the amount of biomass that was growing. The algal biomass that was grown in the algal growth pond system was pumped to the harvester, where the algal biomass was removed, and the tails stream was recycled to the algal aquaculture pretreatment unit.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment. That said, it is understood that any one or more features disclosed herein may be combined.

As used herein, a plurality of items, structural elements, compositional elements, and/or materials may be presented in a common list for convenience. However, these lists should be construed as though each member of the list is individually identified as a separate and unique member. Thus, no individual member of such a list should be construed as a de facto equivalent of any other member of the same list solely based on their presentation in a common group without indications to the contrary. In addition, various embodiments and examples of the present invention may be referred to herein along with alternatives for the various components thereof. It is understood that such embodiments, examples, and alternatives are not to be construed as de facto equivalents of one another but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the foregoing example(s) are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

### REFERENCES CITED

### U.S. Patent Documents

U.S. Pat. No. 5,951,875 September, 1999 Kanel et al.
U.S. Pat. No. 1,851,446 March, 1932 Ranney
U.S. Pat. No. 3,261,401 July, 1966 Karr
U.S. patent Ser. No. 11/599,495 November, 2006 Jones et al.
U.S. patent Ser. No. 11/599,498 November, 2006 Jones et al.
U.S. Pat. No. 4,958,460 September, 1990 Neilson et al.
U.S. Pat. No. 6,986,323 January, 2006 Ayers
U.S. Pat. No. 7,662,616 February, 2010 Hazlebeck, et al.

### FOREIGN PATENT DOCUMENTS

WO/2009/111516 A2 Stabilization of omega-3-fatty acids in oil-water emulsions

### OTHER PUBLICATIONS

Glover, W. B., “Selecting Evaporators for Process Applications”, Chemical Engineering Progress, pp. 26-33, December (2004).
Bear, J. (1972). Dynamics of Fluids in Porous Media. Dover Publications.
Summerhayes, C. P., and Thorpe, S. A., 1996: Oceanography An Illustrated Guide, Chapter 11, pp 165-181.
Andersen, Arthur, “Algal Culturing Techniques,” ISBN 0-12-088426-7, Elsevier Academic Press, Burlington, MA, p. 197 (2005).
Avron and BenAmotz in “Dunaliella: Physiology, Biochemistry, and Biotechnology”, Chapter 4, CRC Press, Boca Raton, FL (1992)
Borowitzka, M. A. “Technical Resource Papers Regional Workshop on the Culture and Utilization of Seaweeds, Volume II,” Fisheries and Aquaculture Department, 1990.

1. A process for preparing and delivering a conditioned aqueous medium to one or more algal aquaculture ponds, wherein the process comprises:
conditioning an aqueous medium with at least algal nutrients in an aquaculture pretreatment unit to obtain the conditioned aqueous medium, the conditioned aqueous medium capable of promoting growth of algae therein;
controlling a gravity flow of the conditioned aqueous medium from the aquaculture pretreatment unit to one or more algal aquaculture ponds; and
modifying a salinity of the conditioned aqueous medium prior to delivery of the conditioned aqueous medium to the one or more algal aquaculture ponds.

2. The process of claim 1, wherein the aqueous medium to be conditioned comprises algal cells.

3. The process of claim 1, wherein the controlling of the gravity flow is done via one or more flow controllers that control flow of the conditioned aqueous medium to the one or more algal aquaculture ponds, wherein optionally the one or more flow controllers comprise one or more weirs, and optionally wherein the one or more weirs comprise a member selected from the group consisting of an overflow weir, an underflow or submerged weir, a flow control valve, and combinations thereof.

4. The process of claim 1, further comprising filtering the aqueous medium or the conditioned aqueous medium prior to gravity flow of the conditioned aqueous medium to the one or more algal aquaculture ponds or the plurality of algal aquaculture ponds.

5. The process of claim 1, wherein the aquaculture pretreatment unit is elevated relative to the one or more algal aquaculture ponds or has a greater surface elevation than the liquid surface of the one or more algal aquaculture ponds to allow for the gravity flow from the aquaculture pretreatment unit to the one or more algal aquaculture ponds.

6. The process of claim 1, further comprising modifying a pH of the conditioned aqueous medium prior to delivery of the conditioned aqueous medium to the one or more algal aquaculture ponds.

7. The process of claim 1, further comprising recycling a used conditioned aqueous medium from the one or more algal aquaculture ponds or from one or more harvesters located downstream of and in fluid communication with the one or more algal aquaculture ponds for the conditioning step.

8. The process of claim 1, wherein the used conditioned aqueous medium is combined with fresh aqueous medium and the resulting aqueous medium is subjected to the conditioning step.

9. The process of claim 1, wherein the process further comprises causing turbulent mixing of the aqueous medium with the algal nutrients in the aquaculture pretreatment unit to provide the conditioned aqueous medium.

10. The process of claim 9, wherein the turbulent mixing of the aqueous medium with the algal nutrients is caused by adding the algal nutrients via a plurality of independent nutrient input streams to the aqueous medium as the aqueous medium flows through the aquaculture pretreatment unit.

11. The process of claim 1, further comprising adding a member selected from the group consisting of seawater, recycled aqueous medium, used conditioned aqueous medium, and inoculum to the aqueous medium in the aquaculture pretreatment unit to generate the conditioned aqueous medium.

12. The process of claim 11, wherein the adding of a member selected from the group consisting of seawater, recycled aqueous medium, used conditioned aqueous medium, and inoculum causes turbulent mixing of the added member with the aqueous medium.

13. The process of claim 1, wherein the aqueous medium comprises a salinity, and wherein the aqueous medium is obtained from one or more of a recycled stream, a natural water source, and a man-made reservoir.

14. The process of claim 1, wherein seawater is accumulated in the aquaculture pretreatment unit during elevated tidal periods.

15. The process of claim 1, further comprising reducing an amount of algal competitors and/or algal predators in the aqueous medium or the conditioned aqueous medium by filtration, addition of one or more oxidizing agents, and/or use of gamma irradiation.

16. The process of claim 1, wherein the aqueous medium is conditioned with a used algal aquaculture medium obtained from a bionutrient recovery facility.

17. An algal aquaculture system comprising:
one or more algal aquaculture ponds;
an aquaculture pretreatment unit configured for conditioning an aqueous medium with at least algal nutrients, wherein the aquaculture pretreatment unit is arranged to feed the conditioned aqueous medium to the one or more algal aquaculture ponds by gravity feed; and
a source of a salinity in fluid communication with the aquaculture pretreatment unit to increase a salinity of the aqueous medium or conditioned aqueous medium.

18. The system of claim 17, wherein the system comprises one or more flow controllers arranged to control the feed of the conditioned aqueous medium from the aquaculture pretreatment unit to the one or more algal aquaculture ponds, optionally wherein the one or more flow controllers comprise one or more weirs, and optionally wherein the one or more weirs comprise selected member from the group consisting of an overflow weir, an underflow or submerged weir, a flow control valve, and combinations thereof.

19. The system of claim 17, wherein the aquaculture pretreatment unit is in fluid communication with a source of the algal nutrients, and wherein the source of the algal nutrients is arranged to cause turbulent mixing of the aqueous medium with the algal nutrients to form the conditioned aqueous medium upon addition to the aquaculture pretreatment unit.

20. The system of claim 17, wherein the source of the algal nutrients comprises a plurality of independent nutrient input streams for adding the algal nutrients to the aquaculture pretreatment unit, and wherein the plurality of independent nutrient input streams comprises independent input streams for at least two of nitrogen, iron, and phosphorus.

21. The system of claim 17, wherein the system further comprises a source of a pH modifier in fluid communication with the aquaculture pretreatment unit to increase pH of the aqueous medium or conditioned aqueous medium.

22. The system of claim 17 wherein the source of additional salinity and/or pH is configured to create turbulent mixing between the aqueous medium or conditioned aqueous medium and the additional salinity and/or pH modifier upon addition to the aquaculture pretreatment unit.

23. The system of claim 17, wherein the system comprises a plurality of algal aquaculture ponds.

24. The system of claim 17, wherein the aquaculture pretreatment unit, the one or more algal aquaculture ponds, and/or the one or more flow controllers of the system comprise a barrier to water permeation.

25. The system of claim 17, wherein the salinity of the conditioned aqueous medium in the aquaculture pretreatment unit is equal to the salinity of the medium in the one or more algal aquaculture ponds.

26. The system of claim 17, wherein the salinity of the conditioned aqueous medium in the aquaculture pretreatment unit and/or in the one or more algal aquaculture ponds is at least about 7 wt-%, at least about 8 wt-%, at least about 9 wt-%, at least about 10 wt-%, at least about 11 wt-%, at least about 12 wt-%, at least about 13 wt-%, at least about 14 wt-%, at least about 15 wt-%, at least about 16 wt-%, at least about 17 wt-%, at least about 18 wt-%, at least about 19 wt-%, at least about 20 wt-%, at least about 21 wt-%, at least about 22 wt-%, at least about 23 wt-%, at least about 24 wt-%, or at least about 25 wt-%.

27. The system of claim 17, wherein the algae comprises or is selected from one or more microalgal species selected from the group consisting of Amphora sp., Anabaena sp., Anabaena flos-aquae, Ankistrodesmus falcatus, Arthrospira sp., Arthrospira (Spirulina) obliquus, Arthrospira (Spirulina) platensis, Botryococcus braunii, Ceramium sp., Chaetoceros gracilis, Chlamydomonas sp., Chlamydomonas mexicana, Chlamydomonas reinhardtii, Chlorella sp., Chlorella fusca, Chlorella protothecoides, Chlorella pyrenoidosa, Chlorella stigmataphora, Chlorella vulgaris, Chlorella zofingiensis, Chlorococcum citriforme, Chlorococcum littorale, Closterium sp., Coccolithus huxleyi, Cosmarium sp., Crypthecoddinium cohnii, Cryptomonas sp., Cyclotella cryptica, Cyclotella nana, Dunaliella sp., Dunaliella bardawil, Dunaliella salina, Dunaliella tertiolecta, Dunaliella viridis, Euglena gracilis, Fragilaria, Fragilaria sublinearis, Gracilaria, Haematococcus pluvialis, Hantzschia, Isochrysis galbana, Microcystis sp., Monochrysis lutheri, Muriellopsis sp., Nannochloris sp., Nannochloropsis sp., Nannochloropsis salina, Navicula sp., Navicula saprophila, Neochloris oleoabundans, Neospongiococcum gelatinosum, Nitzschia laevis, Nitzschia alba, Nitzschia communis, Nitzschia paleacea, Nitzschia closterium, Nitzschia palea, Nostoc commune, Nostoc flagellaforme, Pavlova gyrens, Peridinium, Phaeodactylum tricornutum, Pleurochrysis carterae, Porphyra sp., Porphyridium aerugineum, Porphyridium cruentum, Prymnesium, Prymnesium paruum, Pseudochoricystis ellipsoidea, Rhodomonas sp., Scenedesmus sp., Scenedesmus braziliensis, Scenedesmus obliquus, Scenedesmus quadricauda, Scenedesmus acutus, Scenedesmus dimorphus, Schizochytrium sp., Scytonema, Skeletonema costatum, Spirogyra, Schiochytrium limacinum, Stichococcus bacillaris, Synechoccus, Tetraselmis sp., Tolypothrix sp., genetically-engineered varieties thereof, and any combinations thereof; or one or more prokaryotes selected from the group consisting of Aphanothece halophytica, Microcoleus chthonoplastes, M. lyngbyaceus, Spirulina major, S. platensis, Nodularia spumigena, Dactylococcopsis salina, Synechocystis DUN52, PCC 6803, Synechococcus PCC 7418, Phormidium spp., Oscillatoria spp., Lyngbya spp., Halospirulina tapeticola, Microcystis spp., Nostoc spp., and Aphanocapsa spp.; or one or more eukaryotes selected from the group consisting of Dunaliella spp., Dangeardinella saltitrix, Chlorella vulgaris, Navicula spp., and Amphora spp.; or genetically-engineered varieties of any of the above; or any combinations thereof.

28. The system of claim 17, wherein the system comprises a plurality of algal aquaculture ponds, and wherein the aquaculture pretreatment unit comprises one or more segmented regions to direct or prevent flow of the conditioned aqueous medium into selected ones of the plurality of algal aquaculture ponds.

29. The system of claim 17, wherein the system is configured for the process.

30. Use of the system of claim 17 for conditioning aqueous medium for the growth of algae, treating an algal aquaculture, and/or culturing algae.
